(19) United States
(12) Patent Application Publication
(10) Pub. No.: US 2008/0299550 A1
(43) Pub. Date: Dec. 4, 2008

(54) Methods and Kits For the Prediction of Therapeutic Success and Recurrence Free Survival In Cancer Therapy

(75) Inventors: Marc Munnes, Erkrath (DE); Volkmar Muller, Hamburg (DE); Michael Bader, Hamburg (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: BAYER HEALTHCARE AG, LEVERKUSEN GERMANY (DE)

(21) Appl. No.: 11/664,380

(22) PCT Filed: Sep. 22, 2005
(86) PCT No.: PCT/EP05/10262

§ 371 Date: Jan. 22, 2008

(30) Foreign Application Priority Data
Sep. 20, 2004 (EP) 04023273.8

(51) Int. Cl. C12Q 1/68

(52) U.S. Cl. 435/6

(57) ABSTRACT

The invention provides novel compositions, methods and uses, for the prediction, diagnosis, prognosis, prevention and treatment of malignant neoplasia and breast cancer. The invention further relates to genes that are differentially expressed in breast tissue of breast cancer patients versus those of normal “healthy” tissue. Differentially expressed genes for the identification of patients which are likely to respond to chemotherapy are also provided.

### TECHNICAL FIELD OF THE INVENTION

The present invention relates to methods for the prediction of therapeutic success in cancer therapy. In a preferred embodiment of the invention it relates to methods for prediction of therapeutic success in CMF (cyclophosphamide/methotrexate/fluorouracil) chemotherapy. The methods of the invention are based on determination of expression levels of 84 human genes which are differentially expressed prior to the onset of anti-cancer chemotherapy. The methods and compositions of the invention are most useful in the investigation of breast cancer and CMF therapy, but are useful in the investigation of other types of cancer and therapies as well.

### BACKGROUND OF THE INVENTION AND PRIOR ART

Cancer is the second leading cause of death in the United States after cardiovascular disease. One in three Americans will develop cancer in his or her lifetime, and one of every four Americans will die of cancer. More specifically breast cancer claims the lives of approximately 40,000 women and is diagnosed in approximately 200,000 women annually in the United States alone. Tumors in general are classified based on different parameters, such as tumor size, invasion status, involvement of lymph nodes, metastasis, histolopathology, immunohistochemical markers, and molecular markers (WHO. International Classification of diseases (1); Sabin and Wittekind, 1997 (2)). With the recent advances in gene chip technology, researchers are increasingly focusing on the categorization of tumors based on the distinct expression of marker genes Sorlie et al., 2001 (3): van 't Veer et al., 2002 (4).

It is a well established fact, that adjuvant systemic treatment after surgery reduces the risk of disease relapse and death in patients with primary operable breast cancer. In general, all patients of a given cohort do receive the same treatment, even though many will fail in treatment success. Bio-markers reflecting the tumor response can function as sensitive short-term surrogates of long-term outcome. The use of such bio-markers will make chemotherapy more effective for the individual patient and will allow to change regimen early in case of the non responding tumors.

Although much effort has been made to develop an optimal clinical treatment course for an individual patient with breast cancer, only little progress could be achieved predicting the individual's response to a certain therapy. Such predictions are usually based on standard clinical parameters such as tumor stage and grade, estrogen (ER) and progesterone (PgR) receptors' status, growth rate, over-expression of the HER2/neu and p53 oncogenes. However, evidences about association of ER and/or PgR gene expression with outcome prediction for adjuvant endocrine chemotherapy are still controversial. Studies have shown that levels of ER and PgR gene expression of breast cancer patients are of prognostic importance independently from a subsequent adjuvant chemotherapy. From the theoretical point of view, it is unexpected that the therapeutic response in patients with breast cancer might be independent from the ER/PgR status. It is more probable that the prognostic impact of receptors' expression depends on the impact of other parameters, for example of the ERBB2 receptor. It causes problems finding such factors using conventional biological techniques because all these analyses survey one gene at a time.

Researchers are increasingly focusing on the categorization of tumors based on the distinct expression of marker genes and the DNA microarray technology has been very useful for quantitative measurements of expression levels of thousands of genes simultaneously in one sample. So far this technology has been applied for the classification of cancer tissues e.g., breast tumors [(3), (22-2683)], prediction of metastasis and patient's outcome [(4), (27-29)], and tumor response to chemotherapy [(30-33)].

But nevertheless chemotherapy remains a mainstay in therapeutic regimens offered to patients with breast cancer, particularly those who have cancer that has metastasized from its site of origin [Perez, 1999, (5)]. There are several chemotherapeutic agents that have demonstrated activity in the treatment of breast cancer and research is continuously in an attempt to determine optimal drugs and regimens. However, different patients tend to respond differently to the same therapeutic regimen. Currently, the individuals response to certain therapy can only be assessed statistically, based on data of former clinical studies. There are still a great number of patients who will not benefit from a systemic chemotherapy. Especially, breast cancers are very heterogeneous in their aggressiveness and treatment response. They contain different genetic mutations and variations affecting growth characteristic and sensitivity to several drugs. Identification of each tumor's molecular fingerprint, then, could help to segregate patients who have particularly aggressive tumors or who need to be treated with specific beneficial therapies. As research involving genetics and associated responses to treatment matures, standard practice will undoubtedly become more individualized, enabling physicians to provide specific treatment regimens matched with a tumor's genetic profiles to ensure optimal outcomes. As an alternative therapeutic concept neoadjuvant or primary systemic therapy (PST) can be offered to those patients with either larger inoperable breast cancers or to patients interested in breast conserving surgery (91). The PST in general do not offer a survival advantage over standard adjuvant treatment, but may identify patients with a pathologically confirmed complete response (CR). In this therapeutic setting such biomarkers capable of predict response can be measured in vivo by correlating gene expression directly to the tumor response.

### SUMMARY OF THE INVENTION

The present invention is based on the unexpected finding, that 84 human genes are differentially expressed in neoplastic tissue of patients responding well to adjuvant CMF chemotherapy as compared to patients not responding well to adjuvant CMF chemotherapy. Response to an adjuvant systemic therapy may be the prolonged recurrence free survival time after intervention for the primary tumor, but may also reflect the over all survival time. Hence, elevated or decreased levels of expression in one or several of the 84 genes at the time of tumor surgery or prior to any intervention (e.g. punch biopsy sample) was found to provide valuable information on whether or not a patient is likely to develop distant metastasis despite the given mode of chemotherapy. This would also imply, that those individuals predicted to not develop distant metastasis within a given time frame (e.g. 5 years) will benefit from such chemotherapy regimen and their tumors do respond to the drugs. In a preferred embodiment of the invention, said given mode of chemotherapy is CMF chemotherapy.

The present invention relates to 84 human genes, which are differentially expressed in neoplastic tissue of patients responding well to adjuvant CMF chemotherapy as compared to patients not responding well to adjuvant CMF chemotherapy as determined by the onset of distant metastasis in the non responding cohort.

The present invention furthermore relates to methods of investigating the response of a patient to anti-cancer chemotherapy by determination of the differential expression of one or several genes of a group of 84 human genes, at the time of tumor excision and before the onset of anti-cancer chemotherapy in a patient. Said investigation of the response can be performed immediately after surgery or at time of first biopsy, at a stage in which other methods can not provide the required information on the patient's response to chemotherapy.

Hence the current invention provides means to decide—shortly after tumor surgery—whether or not a certain mode of chemotherapy is likely to be beneficial to the patient's health and/or whether to maintain or change the applied mode of chemotherapy treatment.

The present invention relates to the identification of 84 human genes being differentially expressed in neoplastic tissue resulting in an altered clinical behavior of a neoplastic lesion. The differential expression of these 84 genes is not limited to a specific neoplastic lesion in a certain tissue of the human body.

Genes undergoing expressional changes as response to a chemotherapeutic agent, can serve further on as monitoring markers for the therapy and, if they do correlate with the clinical outcome, such genes may also work as efficacy biomarkers.

In preferred embodiments of this invention the neoplastic lesion is breast cancer. This cancer is not limited to females and may also be diagnosed and analyzed in males.

The invention relates to various methods, reagents and kits for the prediction of therapeutic success in the therapy of breast cancer. “Breast cancer” as used herein includes carcinomas, (e.g., carcinoma in situ, invasive carcinoma, metastatic carcinoma) and pre-malignant conditions, neomorphic changes independent of their histological origin (e.g. ductal, lobular, medullary, mixed origin). The compositions, methods, and kits of the present invention comprise comparing the level of mRNA expression of a single or plurality (e.g. 2, 5, 10, or 50 or more) of genes (hereinafter “marker genes”, listed in Table 1, and the respective polypeptide sequences coded by them) in a patient sample, and the average level of expression of the marker gene(s) in a sample from a control subject (e.g., a human subject without breast cancer). Comparison of the expression level of one or several marker genes can also be performed on any other reference (e.g. tissue samples from responding tumors).

The invention relates further to various compositions, methods, reagents and kits, for prediction of clinically measurable tumor therapy response to a given breast cancer therapy. The compositions, methods of the present invention comprise comparing the level of mRNA expression of a single or plurality (e.g. 2, 5, 10, or 50 or more) of breast cancer marker genes in an unclassified patient sample, and the average level of expression of the marker gene(s) in a sample cohort comprising patient responding in different intensity to an administered adjuvant breast cancer therapy. In preferred embodiments of this invention the specific expression of the marker genes can be utilized for discrimination of responders and non-responders to an CMF based chemotherapeutic intervention.

In further preferred embodiments, the control level of mRNA expression is the average level of expression of the marker gene(s) in samples from several (e.g., 2, 4, 8, 10, 15, 30 or 50) control subjects. These control subjects may also be affected by breast cancer and be classified by their clinical and not necessarily by their individual expression profile.

As elaborated below, a significant change in the level of expression of one or more of the marker genes (set of marker genes) in the patient sample relative to the control level provides significant information regarding the patient's breast cancer status and responsiveness to chemotherapy, preferably CMF chemotherapy. In the compositions, methods, and kits of the present invention the marker genes listed in Table 1 may also be used in combination with well known breast cancer marker genes (e.g. CEA, mammaglobin, or CA 15-3).

According to the invention, the marker gene(s) and marker gene sets are selected such that the positive predictive value of the compositions, methods, and kits of the invention is at least about 10%, preferably about 25%, more preferably about 50% and most preferably about 90% in any of the following conditions: stage 0 breast cancer patients, stage I breast cancer patients, stage II breast cancer patients, stage III breast cancer patients, stage IV breast cancer patients, grade I breast cancer patients, grade II breast cancer patients, grade III breast cancer patients, malignant breast cancer patients, patients with primary carcinomas of the breast, and all other types of cancers, malignancies and transformations associated with the breast.

The detection of marker gene expression is not limited to the detection within a primary, secondary or metastatic lesion of breast cancer patients, and may also be detected in lymph nodes affected by breast cancer cells or minimal residual disease cells either locally deposited (e.g. bone marrow, liver, kidney) or freely floating throughout the patients body.

In one embodiment of the compositions, methods, reagents and kits of the present invention, the sample to be analyzed is tissue material from neoplastic lesion taken by aspiration or punctuation, excision or by any other surgical method leading to biopsy or resected cellular material. In one embodiment of the compositions, methods, and kits of the present invention, the sample comprises cells obtained from the patient. The cells may be found in a breast cell “smear” collected, for example, by a nipple aspiration, ductal lavarge, fine needle biopsy or from provoked or spontaneous nipple discharge. In another embodiment, the sample is a body fluid. Such fluids include, for example, blood fluids, lymph, ascitic fluids, gynecological fluids, or urine but not limited to these fluids.

In accordance with the compositions, methods, and kits of the present invention the determination of gene expression is not limited to any specific method or to the detection of mRNA. The presence and/or level of expression of the marker gene in a sample can be assessed, for example, by measuring and/or quantifying of:

1) a protein encoded by the marker gene in Table 1 or a protein comprising a polypeptide corresponding to a marker gene in Table 1 or a polypeptide resulting from processing or degradation of the protein (e.g. using a reagent, such as an antibody, an antibody derivative, or an antibody fragment, which binds specifically with the protein or polypeptide)
2) a metabolite which is produced directly (i.e., catalyzed) or indirectly by a protein encoded by the marker gene in Table 1 or by a polypeptide encoded thereby.
3) a RNA transcript (e.g., mRNA, hnRNA) encoded by the marker gene in Table 1, or a fragment of the RNA transcript (e.g. by contacting a mixture of RNA transcripts obtained from the sample or cDNA prepared from the transcripts with a substrate having nucleic acid comprising a sequence of one or more of the marker genes listed within Table 1 fixed thereto at selected positions). The mRNA expression of these genes can be detected e.g. with DNA-microarray as provided by Affymetrix Inc. or other manufacturers (U.S. Pat. No. 5,556,752). In a further embodiment the expression of these genes can be detected with bead based direct fluorescent readout techniques such as provided by Luminex Inc. (WO 97/14028).

The composition, method, and kit of the present invention is particularly useful for identifying patients who will not respond to a certain chemotherapy and therefor develop recurrent disease. For this purpose the composition, method, and kit comprises comparing

a) the level of expression of a single or plurality of marker genes in a patient sample, wherein at least one (e.g. 2, 5, 10, or 50 or more) of the marker genes is selected from the marker genes of Table 1 and
b) the level of expression of the marker gene in a control subject or any other reference expression pattern. The control subject may either be not affected by breast cancer or be identified and classified by their clinical response to the particular chemotherapy.

It will be appreciated that in this composition, method, and kit the “therapy” may be any therapy for treating breast cancer including, but not limited to, chemotherapy, anti-hormonal therapy, directed antibody therapy, radiation therapy and surgical removal of tissue, e.g., a breast tumor. Thus, the compositions, methods, and kits of the invention may be used to evaluate a patient before, during and after therapy, for example, to evaluate the reduction in tumor burden.

In another aspect, the invention provides a composition, method, and kit for in vitro selection of a therapy regime (e.g. the kind of chemotherapeutic argents) for inhibiting breast cancer in a patient. This composition, method, and kit comprises the steps of:

a) obtaining a sample comprising cancer cells from the patient;
b) separately maintaining aliquots of the sample in the presence of a diverse test compositions;
c) comparing expression of a single or plurality of marker genes, selected from the marker genes listed in Table 1;
in each of the aliquots; and
d) selecting one of the test compositions which induces a lower level of expression of genes from Table 1 and/or a higher level of expression of genes from Table 1 in the aliquot containing that test composition, relative to the level of expression of each marker gene in the aliquots containing the other test compositions.

The invention further provides a composition, method, and kit of making an isolated hybridoma which produces an antibody useful for assessing whether a patient is afflicted with breast cancer. The composition, method, and kit comprises isolating a protein encoded by a marker gene listed within Table 1 or a polypeptide fragment of the protein, immunizing a mammal using the isolated protein or polypeptide fragment, isolating splenocytes from the immunized mammal, fusing the isolated splenocytes with an immortalized cell line to form hybridomas, and screening individual hybridomas for production of an antibody which specifically binds with the protein or polypeptide fragment to isolate the hybridoma. The invention also includes an antibody produced by this method. Such antibodies specifically bind to a full-length or partial polypeptide comprising a polypeptide listed in Table 1.

The invention also provides various kits. Such kit comprises reagents for assessing expression of a single or a plurality of genes selected from the marker genes listed in Table 1.

In an additional aspect, the invention provides a kit for assessing the presence of breast cancer cells. This kit comprises an antibody, wherein the antibody binds specifically with a protein encoded by a marker gene listed within Table 1 or polypeptide fragment of the protein. The kit may also comprise a plurality of antibodies, wherein the plurality binds specifically with-the protein encoded by each marker gene of a marker gene set listed in Table 1.

In yet another aspect, the invention provides a kit for assessing the presence of breast cancer cells, wherein the kit comprises a nucleic acid probe. The probe hybridizes specifically with a RNA transcript of a marker gene listed within Table 1 or cDNA of the transcript. The kit may also comprise a plurality of probes, wherein each of the probes hybridizes specifically with a RNA transcript of one of the marker genes of a marker gene set listed in Table 1.

It will be appreciated that the compositions, methods, and kits of the present invention may also include known cancer marker genes including known breast cancer marker genes. It will further be appreciated that the compositions, methods, and kits may be used to identify cancers other than breast cancer.

### DETAILED DESCRIPTION OF THE INVENTION

### Definitions

“Differential expression”, or “expression” as used herein, refers to both quantitative as well as qualitative differences in the genes' expression patterns observed in at least two different individuals or samples taken from individuals. Differential expression may depend on differential development, different genetic background of tumor cells and/or reaction to the tissue environment of the tumor. Differentially expressed genes may represent “marker genes,” and/or “target genes”. The expression pattern of a differentially expressed gene disclosed herein may be utilized as part of a prognostic or diagnostic breast cancer evaluation.

The term “pattern of expression” refers, e.g., to a determined level of gene expression compared either to a reference gene (e.g. housekeeper) or to a computed average expression value (e.g. in DNA-chip analyses). A pattern is not limited to the comparison of two genes but even more related to multiple comparisons of genes to a reference genes or samples. A certain “pattern of expression” may also result and be determined by comparison and measurement of several genes disclosed hereafter and display the relative abundance of these transcripts to each other.

Alternatively, a differentially expressed gene disclosed herein may be used in methods for identifying reagents and compounds and uses of these reagents and compounds for the treatment of breast cancer as well as methods of treatment. The differential regulation of the gene is not limited to a specific cancer cell type or clone, but rather displays the interplay of cancer cells, muscle cells, stromal cells, connective tissue cells, other epithelial cells, endothelial cells and blood vessels as well as cells of the immune system (e.g. lymphocytes, macrophages, killer cells).

A “reference pattern of expression levels”, within the meaning of the invention shall be understood as being any pattern of expression levels that can be used for the comparison to another pattern of expression levels. In a preferred embodiment of the invention, a reference pattern of expression levels is, e.g., an average pattern of expression levels observed in a group of healthy or diseased individuals, serving as a reference group.

“Primer pairs and probes”, within the meaning of the invention, shall have the ordinary meaning of this term which is well known to the person skilled in the art of molecular biology. In a preferred embodiment of the invention “primer pairs and probes”, shall be understood as being polynucleotide molecules having a sequence identical, complementary, homologous, or homologous to the complement of regions of a target polynucleotide which is to be detected or quantified.

“Individually labeled probes”, within the meaning of the invention, shall be understood as being molecular probes comprising a polynucleotide or oligonucleotide and a label, helpful in the detection or quantification of the probe. Preferred labels are fluorescent labels, luminescent labels, radioactive labels and dyes.

“Arrayed probes”, within the meaning of the invention, shall be understood as being a collection of immobilized probes, preferably in an orderly arrangement. In a preferred embodiment of the invention, the individual “arrayed probes” can be identified by their respective position on the solid support, e.g., on a “chip”.

The phrase “tumor response”, “therapeutic success”, or “response to therapy” refers, in the adjuvant chemotherapeutic setting to the observation of a defined tumor free or recurrence free survival time (e.g. 2 years, 4 years, 5 years, 10 years). This time period of disease free survival may vary among the different tumor entities but is sufficiently longer than the average time period in which most of the recurrences appear. In a neoadjuvant therapy modality response may be monitored by measurement of tumor shrinkage due to apoptosis and necrosis of the tumor mass.

The term “recurrence” or “recurrent disease” does include distant metastasis that can appear even many years after the initial diagnosis and therapy of a tumor, or to local events such as infiltration of tumor cell into regional lyph nodes, or occurrence of tumor cells at the same site and organ of origin within an appropriate time.

“Prediction of recurrence” or “prediction of success” does refer to the methods an compositions described in this invention. Wherein a tumor specimen is analyzed for it's gene expression and furthermore classified based on correlation of the expression pattern to known ones from reference samples. This classification may either result in the statement that such given tumor will develop recurrence and therefore is considered as a “non responding” tumor to the given therapy, or may result in a classification as a tumor with a prorogued disease free post therapy time.

“Biological activity” or “bioactivity” or “activity” or “biological function”, which are used interchangeably, herein mean an effector or antigenic function that is directly or indirectly performed by a polypeptide (whether in its native or denatured conformation), or by any fragment thereof in vivo or in vitro. Biological activities include but are not limited to binding to polypeptides, binding to other proteins or molecules, enzymatic activity, signal transduction, activity as a DNA binding protein, as a transcription regulator, ability to bind damaged DNA, etc. A bioactivity can be modulated by directly affecting the subject polypeptide. Alternatively, a bioactivity can be altered by modulating the level of the polypeptide, such as by modulating expression of the corresponding gene.

The term “marker” or “biomarker” refers a biological molecule, e.g., a nucleic acid, peptide, hormone, etc., whose presence or concentration can be detected and correlated with a known condition, such as a disease state.

The term “marker gene,” as used herein, refers to a differentially expressed gene which expression pattern may be utilized as part of predictive, prognostic or diagnostic process in malignant neoplasia or breast cancer evaluation, or which, alternatively, may be used in methods for identifying compounds useful for the treatment or prevention of malignant neoplasia and breast cancer in particular. A marker gene may also have the characteristics of a target gene.

“Target gene”, as used herein, refers to a differentially expressed gene involved in breast cancer in a manner by which modulation of the level of target gene expression or of target gene product activity may act to ameliorate symptoms of malignant neoplasia and breast cancer in particular. A target gene may also have the characteristics of a marker gene.

The term “neoplastic lesion” or “neoplastic disease” or “neoplasia” refers to a cancerous tissue this includes carcinomas, (e.g., carcinoma in situ, invasive carcinoma, metastatic carcinoma) and pre-malignant conditions, neomorphic changes independent of their histological origin (e.g. ductal, lobular, medullary, mixed origin). The term “cancer” is not limited to any stage, grade, histomorphological feature, invasiveness, agressivity or malignancy of an affected tissue or cell aggregation. In particular stage 0 breast cancer, stage I breast cancer, stage II breast cancer, stage III breast cancer, stage IV breast cancer, grade I breast cancer, grade II breast cancer, grade III breast cancer, malignant breast cancer, primary carcinomas of the breast, and all other types of cancers, malignancies and transformations associated with the breast are included. The terms “neoplastic lesion” or “neoplastic disease” or “neoplasia” or “cancer” are not limited to any tissue or cell type they also include primary, secondary or metastatic lesion of cancer patients, and also comprises lymph nodes affected by cancer cells or minimal residual disease cells either locally deposited (e.g. bone marrow, liver, kidney) or freely floating throughout the patients body.

Furthermore, the term “characterizing the sate of a neoplastic disease” is related to, but not limited to, measurements and assessment of one or more of the following conditions: Type of tumor, histomorphological appearance, dependence on external signal (e.g. hormones, growth factors), invasiveness, motility, state by TNM (2) or similar, agressivity, malignancy, metastatic potential, and responsiveness to a given therapy.

The term “biological sample”, as used herein, refers to a sample obtained from an organism or from components (e.g., cells) of an organism. The sample may be of any biological tissue or fluid. Frequently the sample will be a “clinical sample” which is a sample derived from a patient. Such samples include, but are not limited to, sputum, blood, blood cells (e.g., white cells), tissue or fine needle biopsy samples, cell-containing body fluids, free floating nucleic acids, urine, peritoneal fluid, and pleural fluid, or cells therefrom. Biological samples may also include sections of tissues such as frozen or fixed sections taken for histological purposes. A biological sample to be analyzed is tissue material from neoplastic lesion taken by aspiration or punctuation, excision or by any other surgical method leading to biopsy or resected cellular material. Such biological sample may comprises cells obtained from a patient. The cells may be found in a breast cell “smear” collected, for example, by a nipple aspiration, ductal lavarge, fine needle biopsy or from provoked or spontaneous nipple discharge. In another embodiment, the sample is a body fluid. Such fluids include, for example, blood fluids, lymph, ascitic fluids, gynecological fluids, or urine but not limited to these fluids.

The term “therapy modality”, “therapy mode”, “regimen” or “chemo regimen” as well as “therapy regime” refers to a timely sequential or simultaneous administration of anti tumor, and/or immune stimulating, and/or blood cell proliferative agents, and/or radiation therapy, and/or hyperthermia, and/or hypothermia for cancer therapy. The administration of these can be performed in an adjuvant and/or neoadjuvant mode. The composition of such “protocol” may vary in dose of the single agent, timeframe of application and frequency of administration within a defined therapy window. Currently various combinations of various drugs and/or physical methods, and various schedules are under investigation.

By “array” or “matrix” is meant an arrangement of addressable locations or “addresses” on a device. The locations can be arranged in two dimensional arrays, three dimensional arrays, or other matrix formats. The number of locations can range from several to at least hundreds of thousands. Most importantly, each location represents a totally independent reaction site. Arrays include but are not limited to nucleic acid arrays, protein arrays and antibody arrays. A “nucleic acid array” refers to an array containing nucleic acid probes, such as oligonucleotides, polynucleotides or larger portions of genes. The nucleic acid on the array is preferably single stranded. Arrays wherein the probes are oligonucleotides are referred to as “oligonucleotide arrays” or “oligonucleotide chips.” A “microarray,” herein also refers to a “biochip” or “biological chip”, an array of regions having a density of discrete regions of at least about 100/cm², and preferably at least about 1000/cm². The regions in a microarray have typical dimensions, e.g., diameters, in the range of between about 10-250 μm, and are separated from other regions in the array by about the same distance. A “protein array” refers to an array containing polypeptide probes or protein probes which can be in native form or denatured. An “antibody array” refers to an array containing antibodies which include but are not limited to monoclonal antibodies (e.g. from a mouse), chimeric antibodies, humanized antibodies or phage antibodies and single chain antibodies as well as fragments from antibodies.

The term “agonist”, as used herein, is meant to refer to an agent that mimics or upregulates (e.g., potentiates or supplements) the bioactivity of a protein. An agonist can be a wild-type protein or derivative thereof having at least one bioactivity of the wild-type protein. An agonist can also be a compound that upregulates expression of a gene or which increases at least one bioactivity of a protein. An agonist can also be a compound which increases the interaction of a polypeptide with another molecule, e.g., a target peptide or nucleic acid.

The term “antagonist” as used herein is meant to refer to an agent that downregulates (e.g., suppresses or inhibits) at least one bioactivity of a protein. An antagonist can be a compound which inhibits or decreases the interaction between a protein and another molecule, e.g., a target peptide, a ligand or an enzyme substrate. An antagonist can also be a compound that downregulates expression of a gene or which reduces the amount of expressed protein present.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon-containing) or inorganic molecules. Many pharmaceutical companies have extensive libraries of chemical and/or biological mixtures, often fungal, bacterial, or algal extracts, which can be screened with any of the assays of the invention to identify compounds that modulate a bioactivity.

The terms “modulated” or “modulation” or “regulated” or “regulation” and “differentially regulated” as used herein refer to both upregulation (i.e., activation or stimulation (e.g., by agonizing or potentiating) and down regulation [i.e., inhibition or suppression (e.g., by antagonizing, decreasing or inhibiting)].

“Transcriptional regulatory unit” refers to DNA sequences, such as initiation signals, enhancers, and promoters, which induce or control transcription of protein coding sequences with which they are operably linked. In preferred embodiments, transcription of one of the genes is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally occurring forms of the polypeptide.

The term “derivative” refers to the chemical modification of a polypeptide sequence, or a polynucleotide sequence. Chemical modifications of a polynucleotide sequence can include, for example, replacement of hydrogen by an alkyl, acyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived. The term “derivative” furthermore refers to phosphorylated forms of a polypeptide sequence or protein.

The term “nucleotide analog” refers to oligomers or polymers being at least in one feature different from naturally occurring nucleotides, oligonucleotides or polynucleotides, but exhibiting functional features of the respective naturally occurring nucleotides (e.g. base paring, hybridization, coding information) and that can be used for said compositions. The nucleotide analogs can consist of non-naturally occurring bases or polymer backbones, examples of which are LNAs, PNAs and Morpholinos. The nucleotide analog has at least one molecule different from its naturally occurring counterpart or equivalent.

“BREAST CANCER GENES” or “BREAST CANCER GENE” as used herein refers to the polynucleotides Table 1, as well as derivatives, fragments, analogs and homologues thereof, the polypeptides encoded thereby as well as derivatives, fragments, analogs and homologues thereof and the corresponding genomic transcription units which can be derived or identified with standard techniques well known in the art using the information disclosed in Tables 1 to 4. The Gene symbol, Gene Description, Reference, locus link ID, Unigene ID, and OMIM number are shown in Table 1.

The term “kit” as used herein refers to any manufacture (e.g. a diagnostic or research product) comprising at least one reagent, e.g. a probe, for specifically detecting the expression of at least one marker gene disclosed in the invention, in particular of those genes listed in Table 1, whereas the manufacture is being sold, distributed, and/or promoted as a unit for performing the methods of the present invention. Also reagents (e.g. immunoassays) to detect the presence, the stability, activity, complexity of the respective marker gene products comprising polypeptides encoded by the genes listed in Table 1 regard as components of the kit. In addition, any combination of nucleic acid and protein detection as disclosed in the invention are regard as a kit.

The present invention provides polynucleotide sequences and proteins encoded thereby, as well as probes derived from the polynucleotide sequences, antibodies directed to the encoded proteins, and predictive, preventive, diagnostic, prognostic and therapeutic uses for individuals which are at risk for or which have malignant neoplasia and breast cancer in particular. The sequences disclosure herein have been found to be differentially expressed in samples from breast cancer.

The present invention is based on the identification of 84 genes that are differentially regulated (up- or down regulated) in tumor biopsies of patients with clinical evidence of breast cancer. The characterization of the co-expression of some of these genes provides newly identified roles in breast cancer.

It is obvious to the person skilled in the art that a reference to a nucleotide sequence is meant to comprise the reference to the associated protein sequence which is coded by said nucleotide sequence.

“% identity” of a first sequence towards a second sequence, within the meaning of the invention, means the % identity which is calculated as follows: First the optimal global alignment between the two sequences is determined with the CLUSTALW algorithm [Thomson J D, Higgins D G, Gibson T J. 1994. ClustalW: Improving the sensitivity of progressive multiple sequence alignment through sequence weighting, positions-specific gap penalties and weight matrix choice. Nucleic Acids Res., 22: 4673-4680], Version 1.8, applying the following command line syntax: ./clustalw -infile=./infile.txt -output= -outorder=aligned -pwmatrix=gonnet -pwdnamatrix=clustalw -pwgapopen=10.0 -pwgapext=0.1 -matrix=gonnet -gapopen=10.0 -gapext=0.05 -gapdist=8-hgapresidues=GPSNDQERK -maxdiv=40. Implementations of the CLUSTAL W algorithm are readily available at numerous sites on the internet, including, e.g., http://www.ebi.ac.uk. Thereafter, the number of matches in the alignment is determined by counting the number of identical nucleotides (or amino acid residues) in aligned positions. Finally, the total number of matches is divided by the number of nucleotides (or amino acid residues) of the longer of the two sequences, and multiplied by 100 to yield the % identity of the first sequence towards the second sequence.

The present invention relates to:

1. A method for predicting therapeutic success of a given mode of treatment in a subject having breast cancer, comprising
(i) determining the pattern of expression levels of at least 6, 8, 10, 15, 20, 30, or 84 marker genes, comprised in the group of marker genes listed in Table 1,
(ii) comparing the pattern of expression levels determined in (i) with one or several reference pattern(s) of expression levels,
(iii) predicting therapeutic success for said given mode of treatment in said subject from the outcome of the comparison in step (ii).
   2. A method of count 1, wherein said given mode of treatment
(i) acts on cell proliferation, and/or
(ii) acts on cell survival, and/or
(iii) acts on cell motility; and/or
(iv) comprises administration of a chemotherapeutic agent.
   3. A method of count 1 or 2, wherein said given mode of treatment is CMF (cyclophosphamide, methotrexate, fluorouracil) chemotherapy.
   4. A method of any of counts 1 to 3, wherein a predictive algorithm is used.
   5. A method of treatment of a neoplastic disease in a subject, comprising
(i) predicting therapeutic success for a given mode of treatment in a subject having breast cancer by the method of any of counts 1 to 4,
(ii) treating said neoplastic disease in said patient by said mode of treatment, if said mode of treatment is predicted to be successful.
   6. A method of selecting a therapy modality for a subject afflicted with a neoplastic disease, comprising
(i) obtaining a biological sample from said subject,
(ii) predicting from said sample, by the method of any of counts 1 to 4, therapeutic success in a subject having breast cancer for a plurality of individual modes of treatment,
(iii) selecting a mode of treatment which is predicted to be successful in step (ii).
   7. A method of any of counts 1 to 6, wherein the expression level is determined
(i) with a hybridization based method, or
(ii) with a hybridization based method utilizing arrayed probes, or
(iii) with a hybridization based method utilizing individually labeled probes, or
(iv) by real time real time PCR, or
(v) by assessing the expression of polypeptides, proteins or derivatives thereof, or
(vi) by assessing the amount of polypeptides, proteins or derivatives thereof.
   8. A kit comprising at least 6, 8, 10, 15, 20, 30, or 84 primer pairs and probes suitable for marker genes comprised in the group of marker genes listed in Table 1.
   9. A kit comprising at least 6, 8, 10, 15, 20, 30, or 84 individually labeled probes, each having a sequence complementary to any of sequences listed in Table 1.
   10. A kit comprising at least 6, 8, 10, 15, 20, 30, or 84 arrayed probes, each having a sequence complementary to any of the sequences listed in Table 1.

It is apparent to the person skilled in the art that, in order to determine the expression of a gene, parts and fragments of said gene can be used instead.

The invention also relates to methods for determining the probability of successful application of a given mode of treatment in a subject having breast cancer, wherein sequences being homologues to the sequences of Table 1 are used. Preferred homologues have 80, 90, 95, or 99% sequence identity towards the original sequence. Preferably the homologues still have the same biological activity and/or function as have the original molecules.

### Experimental Procedures and Settings

The present invention relates to predicting the successful application of a given mode of treatment to a cancer patient, as those individual will not develop recurrent disease. In a preferred embodiment of the invention, said mode of treatment is CMF (cyclophosphamide, methotrexate, fluorouracil) chemotherapy.

Cyclophosphamide, metothreate and fluorouracil are common therapeutics for advanced and metastatic breast cancer. These compounds have been established as important chemotherapeutic agents in the armamentarium of drugs to treat breast cancer in the 1970s and are still in use. Expression profiles of 56 pre-treatment biopsy samples have been obtained by the use of oligonucleotide microarrays (Affymetrix).

Analyzing the data for 56 by statistical methods as described in EXAMPLES 3 to 5 we identified 84 significantly differentially expressed genes listed in Table 1.

Biological Relevance of the Genes which are Part of the Invention

Some of the genes listed in Table 1 represent biological, cellular processes and are characterized by similar regulation of genes. By the way of illustration but limited to the following examples a few characteristic genes from Table 1 are described in later by greater detail:

### CCNB1

CyclinB is reported to be expressed predominantly in the G2/M phase of cell division. The gene product complexes with p34(cdc2) to form the mitosis-promoting factor (MPF). The multiple cyclin B1-related sequences in the mouse genome and the multiple cyclin B1 mRNAs raised the possibility that the seemingly redundant cyclin B genes may have developmental- and/or cell-type-specific functions.

The human CCNB1 gene map to 5q12, as shown by Southern blot analysis of human/Chinese hamster somatic cell hybrid panels. In vertebrate cells, the nuclear entry of MPF during prophase is thought to be essential for the induction and coordination of M-phase events. Phosphorylation of cyclin B1 is central to its nuclear translocation. During cell cycle progression in HeLa cells, a change in the kinase activity of endogenous PLK1 toward S147 and/or S133 correlated with a kinase activity in the cell extracts. Two B-type cyclins, B1 and B2, have been identified in mammals. Proliferating cells express both cyclins, which bind to and activate p34 (CDC2). To test whether the 2 B-type cyclins have distinct roles, lines of transgenic mice were generated, one lacking cyclin B1 and the other lacking B2. Cyclin B1 proved to be an essential gene; no homozygous B1-null pups were born. These observations suggested that cyclin B1 may compensate for the loss of cyclin B2 in the mutant mice, and implies that cyclin B1 is capable of targeting the p34(CDC2) kinase to the essential substrates of cyclin B2. In higher eukaryotes, the S phase and M phase of the cell cycle are triggered by different cyclin-dependent kinases (CDKs). For example, in frog egg extracts, Cdk1 cyclin B catalyzes entry into mitosis but cannot trigger DNA replication.

### DUSP9

Members of the dual-specificity phosphatase protein family inactivate MAP kinase through dephosphorylation of critical threonine and tyrosine residues. The sequence of the predicted 384-amino acid protein for DUSP 9 is 57% identical to that of DUSP6. Like other dual-specificity phosphatases, the N-terminal regions of DUSP9 contains 2 domains that are homologous to segments known as CH2 domains flanking the active site of the CDC25 phosphatase. In vitro expression of DUSP9 produced a protein with a mass of 41.8 kD by SDS-PAGE. DUSP9 inactivates MAP kinases both in vitro and when expressed in mammalian cells. Like DUSP6, DUSP9 showed selectivity for members of the ERK family of MAP kinases. A punctate nuclear staining pattern, colocalizing with PML was observed in 10 to 20% of cells. Northern blot analysis revealed that DUSP9 is expressed as a 2.5-kb mRNA only in placenta, kidney, and fetal liver.

### RFC4; A1

The elongation of primed DNA templates by DNA polymerase delta and DNA polymerase epsilon requires the action of 2 accessory proteins, proliferating cell nuclear antigen (PCNA) and activator 1. A1 is an enzyme that contains 5 different subunits of 140, 40, 38, 37, and 36 kD. The deduced amino acid sequence showed a high degree of homology to the 40-kD subunit of A1 but, unlike the 40-kD protein, the 37-kD expressed protein did not bind ATP. Other findings suggested that both the 37- and 40-kD subunits of A1 are required for the biologic role of A1 and that they may function differently in this process.

By immunoprecipitation and mass spectrometry analyses one tried to identify BRCA1 associated proteins. One found that BRCA1 is part of a large multisubunit protein complex of tumor suppressors, DNA damage sensors, and signal transducers. They named this complex BASC, for ‘BRCA1-associated genome surveillance complex.’ Among the DNA repair proteins identified in the complex were ATM, BLM, MSH2, MSH6, MLH1, the RAD50 MRE11-NBS1 complex, and the RFC1-RFC2-RFC4 complex. It has been suggested that BASC may serve as a sensor of abnormal DNA structures and/or as a regulator of the postreplication repair process and is involved in cellular replication and proliferation.

### Polynucleotides

A “BREAST CANCER GENE” polynucleotide can be single- or double-stranded and comprises a coding sequence or the complement of a coding sequence for a “BREAST CANCER GENE” polypeptide. Degenerate nucleotide sequences encoding human “BREAST CANCER GENE” polypeptides, as well as homologous nucleotide sequences which are at least about 50, 55, 60, 65, 70, preferably about 75, 90, 96, or 98% identical to the nucleotide sequences of Table 1 also are “BREAST CANCER GENE” polynucleotides.

### Identification of Differential Expression

Transcripts within the collected RNA samples which represent RNA produced by differentially expressed genes may be identified by utilizing a variety of methods which are ell known to those of skill in the art. For example, differential screening [Tedder, T. F. et al., 1988, (8)], subtractive hybridization [Hedrick, S. M. et al., 1984, (9)] and, preferably, differential display (Liang, P., and Pardee, A. B., 1993, U.S. Pat. No. 5,262,311, which is incorporated herein by reference in its entirety), may be utilized to identify polynucleotide sequences derived from genes that are differentially expressed.

Differential screening involves the duplicate screening of a cDNA library in which one copy of the library is screened with a total cell cDNA probe corresponding to the mRNA population of one cell type while a duplicate copy of the cDNA library is screened with a total cDNA probe corresponding to the mRNA population of a second cell type. For example, one cDNA probe may correspond to a total cell cDNA probe of a cell type derived from a control subject, while the second cDNA probe may correspond to a total cell cDNA probe of the same cell type derived from an experimental subject. Those clones which hybridize to one probe but not to the other potentially represent clones derived from genes differentially expressed in the cell type of interest in control versus experimental subjects.

Subtractive hybridization techniques generally involve the isolation of mRNA taken from two different sources, e.g., control and experimental tissue, the hybridization of the mRNA or single-stranded cDNA reverse-transcribed from the isolated mRNA, and the removal of all hybridized, and therefore double-stranded, sequences. The remaining non-hybridized, single-stranded cDNA, potentially represent clones derived from genes that are differentially expressed in the two mRNA sources. Such single-stranded cDNA is then used as the starting material for the construction of a library comprising clones derived from differentially expressed genes.

The differential display technique describes a procedure, utilizing the well known polymerase chain reaction (PCR; the experimental embodiment set forth in Mullis, K. B., 1987, U.S. Pat. No. 4,683,202) which allows for the identification of sequences derived from genes which are differentially expressed. First, isolated RNA is reverse-transcribed into single-stranded cDNA, utilizing standard techniques which are well known to those of skill in the art. Primers for the reverse transcriptase reaction may include, but are not limited to, oligo dT-containing primers, preferably of the reverse primer type of oligonucleotide described below. Next, this technique uses pairs of PCR primers, as described below, which allow for the amplification of clones representing a random subset of the RNA transcripts present within any given cell. Utilizing different pairs of primers allows each of the mRNA transcripts present in a cell to be amplified. Among such amplified transcripts may be identified those which have been produced from differentially expressed genes.

The reverse oligonucleotide primer of the primer pairs may contain an oligo dT stretch of nucleotides, preferably eleven nucleotides long, at its 5′ end, which hybridizes to the poly(A) tail of mRNA or to the complement of a cDNA reverse transcribed from an mRNA poly(A) tail. Second, in order to increase the specificity of the reverse primer, the primer may contain one or more, preferably two, additional nucleotides at its 3′ end. Because, statistically, only a subset of the mRNA derived sequences present in the sample of interest will hybridize to such primers, the additional nucleotides allow the primers to amplify only a subset of the mRNA derived sequences present in the sample of interest. This is preferred in that it allows more accurate and complete visualization and characterization of each of the bands representing amplified sequences.

The forward primer may contain a nucleotide sequence expected, statistically, to have the ability to hybridize to cDNA sequences derived from the tissues of interest. The nucleotide sequence may be an arbitrary one, and the length of the forward oligonucleotide primer may range from about 9 to about 13 nucleotides, with about 10 nucleotides being preferred. Arbitrary primer sequences cause the lengths of the amplified partial cDNAs produced to be variable, thus allowing different clones to be separated by using standard denaturing sequencing gel electrophoresis. PCR reaction conditions should be chosen which optimize amplified product yield and specificity, and, additionally, produce amplified products of lengths which may be resolved utilizing standard gel electrophoresis techniques. Such reaction conditions are well known to those of skill in the art, and important reaction parameters include, for example, length and nucleotide sequence of oligonucleotide primers as discussed above, and annealing and elongation step temperatures and reaction times. The pattern of clones resulting from the reverse transcription and amplification of the mRNA of two different cell types is displayed via sequencing gel electrophoresis and compared. Differences in the two banding patterns indicate potentially differentially expressed genes.

When screening for full-length cDNAs, it is preferable to use libraries that have been size-selected to include larger cDNAs. Randomly-primed libraries are preferable, in that they will contain more sequences which contain the 5′ regions of genes. Use of a randomly primed library may be especially preferable for situations in which an oligo d(T) library does not yield a full-length cDNA. Genomic libraries can be useful for extension of sequence into 5′ nontranscribed regulatory regions.

Commercially available capillary electrophoresis systems can be used to analyze the size or confirm the nucleotide sequence of PCR or sequencing products. For example, capillary sequencing can employ flowable polymers for electrophoretic separation, four different fluorescent dyes (one for each nucleotide) which are laser activated, and detection of the emitted wavelengths by a charge coupled device camera. Output/light intensity can be converted to electrical signal using appropriate software (e.g. GENOTYPER and Sequence NAVIGATOR, Perkin Elmer; ABI), and the entire process from loading of samples to computer analysis and electronic data display can be computer controlled. Capillary electrophoresis is especially preferable for the sequencing of small pieces of DNA which might be present in limited amounts in a particular sample.

Once potentially differentially expressed gene sequences have been identified via bulk techniques such as, for example, those described above, the differential expression of such putatively differentially expressed genes should be corroborated. Corroboration may be accomplished via, for example, such well known techniques as Northern analysis and/or RT-PCR. Upon corroboration, the differentially expressed genes may be further characterized, and may be identified as target and/or marker genes, as discussed, below.

Also, amplified sequences of differentially expressed genes obtained through, for example, differential display may be used to isolate full length clones of the corresponding gene. The full length coding portion of the gene may readily be isolated, without undue experimentation, by molecular biological techniques well known in the art. For example, the isolated differentially expressed amplified fragment may be labeled and used to screen a cDNA library. Alternatively, the labeled fragment may be used to screen a genomic library.

An analysis of the tissue distribution of the mRNA produced by the identified genes may be conducted, utilizing standard techniques well known to those of skill in the art. Such techniques may include, for example, Northern analyses and RT-PCR. Such analyses provide information as to whether the identified genes are expressed in tissues expected to contribute to breast cancer. Such analyses may also provide quantitative information regarding steady state mRNA regulation, yielding data concerning which of the identified genes exhibits a high level of regulation in, preferably, tissues which may be expected to contribute to breast cancer.

Such analyses may also be performed on an isolated cell population of a particular cell type derived from a given tissue. Additionally, standard in situ hybridization techniques may be utilized to provide information regarding which cells within a given tissue express the identified gene. Such analyses may provide information regarding the biological function of an identified gene relative to breast cancer in instances wherein only a subset of the cells within the tissue is thought to be relevant to breast cancer.

### Identification of Polynucleotide Variants and Homologues or Splice Variants

Variants and homologues of the “BREAST CANCER GENE” polynucleotides described above also are “BREAST CANCER GENE” polynucleotides. Typically, homologous “BREAST CANCER GENE” polynucleotide sequences can be identified by hybridization of candidate polynucleotides to known “BREAST CANCER GENE” polynucleotides under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50 EC once, 30 minutes; then 2×SSC, room temperature twice, 10 minutes each homologous sequences can be identified which contain at most about 25-30% basepair mismatches. More preferably, homologous polynucleotide strands contain 15-25% basepair mismatches, even more preferably 5-15% basepair mismatches.

Species homologues of the “BREAST CANCER GENE” polynucleotides disclosed herein also can be identified by making suitable probes or primers and screening cDNA expression libraries from other species, such as mice, monkeys, or yeast. Human variants of “BREAST CANCER GENE” polynucleotides can be identified, for example, by screening human cDNA expression libraries. It is well known that the Tₘ of a double-stranded DNA decreases by 1-1.5° C. with every 1% decrease in homology [Bonner et al., 1973, (10)]. Variants of human “BREAST CANCER GENE” polynucleotides or “BREAST CANCER GENE” polynucleotides of other species can therefore be identified by hybridizing a putative homologous “BREAST CANCER GENE” polynucleotide with a polynucleotide having a nucleotide sequence of one of the genes of the Table 1 or the complement thereof to form a test hybrid. The melting temperature of the test hybrid is compared with the melting temperature of a hybrid comprising polynucleotides having perfectly complementary nucleotide sequences, and the number or percent of basepair mismatches within the test hybrid is calculated.

Nucleotide sequences which hybridize to “BREAST CANCER GENE” polynucleotides or their complements following stringent hybridization and/or wash conditions also are “BREAST CANCER GENE” polynucleotides. Stringent wash conditions are well known and understood in the art and are disclosed, for example, in Sambrook et al., (6), Ausubel (7). Typically, for stringent hybridization conditions a combination of temperature and salt concentration should be chosen that is approximately 12 to 20° C. below the calculated Tₘ of the hybrid under study. The Tₘ of a hybrid between a “BREAST CANCER GENE” polynucleotide having a nucleotide sequence of one of the sequences of Table 1 or the complement thereof and a polynucleotide sequence which is at least about 50, preferably about 75, 90, 96, or 98% identical to one of those nucleotide sequences can be calculated, for example, using the equation below [Bolton and McCarthy, 1962, (11):

Tₘ=81.5° C.-16.6(log₁₀[Na⁺])+0.41(% G+C)−0.63(% formamide)−600/1),

where l=the length of the hybrid in basepairs.

Stringent wash conditions include, for example, 4×SSC at 65° C., or 50% formamide, 4×SSC at 28° C., or 0.5×SSC, 0.1% SDS at 65° C. Highly stringent wash conditions include, for example, 0.2×SSC at 65° C.

### Polypeptides

“BREAST CANCER GENE” polypeptides according to the invention comprise a polypeptide of Table 1 or derivatives, fragments, analogues and homologues thereof. A BREAST CANCER GENE” polypeptide of the invention therefore can be a portion, a full-length, or a fusion protein comprising all or a portion of a “BREAST CANCER GENE” polypeptide.

### Biologically Active Variants

“BREAST CANCER GENE” polypeptide variants which are biologically active, i.e., retain an “BREAST CANCER GENE” activity, can be also regarded as “BREAST CANCER GENE” polypeptides. Preferably, naturally or non-naturally occurring “BREAST CANCER GENE” polypeptide variants have amino acid sequences which are at least about 60, 65, or 70, preferably about 75, 80, 85, 90, 92, 94, 96, or 98% identical to any of the amino acid sequences of the polypeptides of encoded by the genes in Table 1 or the polypeptides encoded by any of the polynucleotides of Table 1 or a fragment thereof.

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of a “BREAST CANCER GENE” polypeptide can be found using computer programs well known in the art, such as DNASTAR software. Whether an amino acid change results in a biologically active “BREAST CANCER GENE” polypeptide can readily be determined by assaying for “BREAST CANCER GENE” activity, as described for example, in the specific Examples, below. Larger insertions or deletions can also be caused by alternative splicing. Protein domains can be inserted or deleted without altering the main activity of the protein.

### Detecting Expression and Gene Product

Although the presence of marker gene expression suggests that the “BREAST CANCER GENE” polynucleotide is also present, its presence and expression may need to be confirmed. For example, if a sequence encoding a “BREAST CANCER GENE” polypeptide is inserted within a marker gene sequence, transformed cells containing sequences which encode a “BREAST CANCER GENE” polypeptide can be identified by the absence of marker gene function. Alternatively, a marker gene can be placed in tandem with a sequence encoding a “BREAST CANCER GENE” polypeptide under the control of a single promoter. Expression of the marker gene in response to induction or selection usually indicates expression of the “BREAST CANCER GENE” polynucleotide.

Alternatively, host cells which contain a “BREAST CANCER GENE” polynucleotide and which express a “BREAST CANCER GENE” polypeptide can be identified by a variety of procedures known to those of skill in the art. These procedures include, but are not limited to, DNA-DNA or DNA-RNA hybridization and protein bioassay or immunoassay techniques which include membrane, solution, or chip-based technologies for the detection and/or quantification of polynucleotide or protein. For example, the presence of a polynucleotide sequence encoding a “BREAST CANCER GENE” polypeptide can be detected by DNA-DNA or DNA-RNA hybridization or amplification using probes or fragments or fragments of polynucleotides encoding a “BREAST CANCER GENE” polypeptide. Nucleic acid amplification-based assays involve the use of oligonucleotides selected from sequences encoding a “BREAST CANCER GENE” polypeptide to detect transformants which contain a “BREAST CANCER GENE” polynucleotide.

A variety of protocols for detecting and measuring the expression of a “BREAST CANCER GENE” polypeptide, using either polyclonal or monoclonal antibodies specific for the polypeptide, are known in the art. Examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on a “BREAST CANCER GENE” polypeptide can be used, or a competitive binding assay can be employed. These and other assays are described in Hampton et al., (12).

A wide variety of labels and conjugation techniques are known by those skilled in the art and can be used in various nucleic acid and amino acid assays. Means for producing labeled hybridization or PCR probes for detecting sequences related to polynucleotides encoding “BREAST CANCER GENE” polypeptides include oligo labeling, nick translation, end-labeling, or PCR amplification using a labeled nucleotide. Alternatively, sequences encoding a “BREAST CANCER GENE” polypeptide can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, magnetic particles, and the like.

### Predictive, Diagnostic and Prognostic Assays

The present invention provides compositions, methods, and kits for determining the probability of successful application of a given mode of treatment in a subject having cancer in particular by detecting the disclosed biomarkers, i.e., the disclosed polynucleotide markers of Table 1.

In clinical applications, biological samples can be screened for the presence and/or absence of the biomarkers identified herein. Such samples are for example needle biopsy cores, surgical resection samples, or body fluids like serum, thin needle nipple aspirates and urine. For example, these methods include obtaining a biopsy, which is optionally fractionated by cryostat sectioning to enrich diseases cells to about 80% of the total cell population. In certain embodiments, polynucleotides extracted from these samples may be amplified using techniques well known in the art. The expression levels of selected markers detected would be compared with statistically valid groups of diseased and healthy samples.

In one embodiment the compositions, methods, and kits comprises determining whether a subject has an abnormal mRNA and/or protein level of the disclosed markers, such as by Northern blot analysis, reverse transcription-polymerase chain reaction (RT-PCR), in situ hybridization, immunoprecipitation, Western blot hybridization, or immunohistochemistry. According to the method, cells are obtained from a subject and the levels of the disclosed biomarkers, protein or mRNA level, is determined and compared to the level of these markers in a healthy subject. An abnormal level of the biomarker polypeptide or mRNA levels is likely to be indicative of malignant neoplasia such as breast cancer.

In another embodiment the compositions, methods, and kits comprises determining whether a subject has an abnormal DNA content of said genes or said genomic loci, such as by Southern blot analysis, dot blot analysis, Fluorescence or Colorimetric In Situ Hybridization, Comparative Genomic Hybridization or quantitative PCR. In general these assays comprise the usage of probes from representative genomic regions. The probes contain at least parts of said genomic regions or sequences complementary or analogous to said regions. In particular intra- or intergenic regions of said genes or genomic regions. The probes can consist of nucleotide sequences or sequences of analogous functions (e.g. PNAs, Morpholino oligomers) being able to bind to target regions by hybridization. In general genomic regions being altered in said patient samples are compared with unaffected control samples (normal tissue from the same or different patients, surrounding unaffected tissue, peripheral blood) or with genomic regions of the same sample that don't have said alterations and can therefore serve as internal controls. In a preferred embodiment regions located on the same chromosome are used. Alternatively, gonosomal regions and/or regions with defined varying amount in the sample are used. In one favored embodiment the DNA content, structure, composition or modification is compared that lie within distinct genomic regions. Especially favored are methods that detect the DNA content of said samples, where the amount of target regions are altered by amplification and or deletions. In another embodiment the target regions are analyzed for the presence of polymorphisms (e.g. Single Nucleotide Polymorphisms or mutations) that affect or predispose the cells in said samples with regard to clinical aspects, being of diagnostic, prognostic or therapeutic value. Preferably, the identification of sequence variations is used to define haplotypes that result in characteristic behavior of said samples with said clinical aspects.

### DNA Array Technology

In one embodiment, the present invention also provides a method wherein polynucleotide probes are immobilized an a DNA chip in an organized array. Oligonucleotides can be bound to a solid support by a variety of processes, including lithography. For example a chip can hold up to 410.000 oligonucleotides (GeneChip, Affymetrix). The present invention provides significant advantages over the available tests for malignant neoplasia, such as breast cancer, because it increases the reliability of the test by providing an array of polynucleotide markers an a single chip.

The method includes obtaining a biological sample which can be a biopsy of an affected person, which is optionally fractionated by cryostat sectioning to enrich diseased cells to about 80% of the total cell population and the use of body fluids such as serum or urine, serum or cell containing liquids (e.g. derived from fine needle aspirates). The DNA or RNA is then extracted, amplified, and analyzed with a DNA chip to determine the presence of absence of the marker polynucleotide sequences. In one embodiment, the polynucleotide probes are spotted onto a substrate in a two-dimensional matrix or array. samples of polynucleotides can be labeled and then hybridized to the probes. Double-stranded polynucleotides, comprising the labeled sample polynucleotides bound to probe polynucleotides, can be detected once the unbound portion of the sample is washed away.

The probe polynucleotides can be spotted on substrates including glass, nitrocellulose, etc. The probes can be bound to the substrate by either covalent bonds or by non-specific interactions, such as hydrophobic interactions. The sample polynucleotides can be labeled using radioactive labels, fluorophores, chromophores, etc. Techniques for constructing arrays and methods of using these arrays are described in EP0 799 897; WO 97/29212; WO 97/27317; EP 0 785 280; WO 97/02357; U.S. Pat. No. 5,593,839; U.S. Pat. No. 5,578,832; EP 0 728 520; U.S. Pat. No. 5,599,695; EP 0 721 016; U.S. Pat. No. 5,556,752; WO 95/22058; and U.S. Pat. No. 5,631,734. Further, arrays can be used to examine differential expression of genes and can be used to determine gene function. For example, arrays of the instant polynucleotide sequences can be used to determine if any of the polynucleotide sequences are differentially expressed between normal cells and diseased cells, for example. High expression of a particular message in a diseased sample, which is not observed in a corresponding normal sample, can indicate a breast cancer specific protein.

Accordingly, in one aspect, the invention provides probes and primers that are specific to the polynucleotide sequences of Table 1.

In one embodiment, the composition, method, and kit comprise using a polynucleotide probe to determine the presence of malignant or breast cancer cells in particular in a tissue from a patient. Specifically, the method comprises:
1) providing a polynucleotide probe comprising a nucleotide sequence at least 12 nucleotides in length, preferably at least 15 nucleotides, more preferably, 25 nucleotides, and most preferably at least 40 nucleotides, and up to all or nearly all of the coding sequence which is complementary to a portion of the coding sequence of a polynucleotide selected from the polynucleotides of Table 1 or a sequence complementary thereto;
2) obtaining a tissue sample from a patient with malignant neoplasia;
3) providing a second tissue sample from a patient with no malignant neoplasia;
4) contacting the polynucleotide probe under stringent conditions with RNA of each of said first and second tissue samples (e.g., in a Northern blot or in situ hybridization assay); and
5) comparing (a) the amount of hybridization of the probe with RNA of the first tissue sample, with (b) the amount of hybridization of the probe with RNA of the second tissue sample;
   wherein a statistically significant difference in the amount of hybridization with the RNA of the first tissue sample as compared to the amount of hybridization with the RNA of the second tissue sample is indicative of malignant neoplasia and breast cancer in particular in the first tissue sample.

### Data Analysis Methods

Comparison of the expression levels of one or more “BREAST CANCER GENES” with reference expression levels, e.g., expression levels in diseased cells of breast cancer or in normal counterpart cells, is preferably conducted using computer systems. In one embodiment, expression levels are obtained in two cells and these two sets of expression levels are introduced into a computer system for comparison. In a preferred embodiment, one set of expression levels is entered into a computer system for comparison with values that are already present in the computer system, or in computer-readable form that is then entered into the computer system.

In one embodiment, the invention provides a computer readable form of the gene expression profile data of the invention, or of values corresponding to the level of expression of at least one “BREAST CANCER GENE” in a diseased cell. The values can be mRNA expression levels obtained from experiments, e.g., microarray analysis. The values can also be mRNA levels normalised relative to a reference gene whose expression is constant in numerous cells under numerous conditions, e.g., GAPDH. In other embodiments, the values in the computer are ratios of, or differences between, normalized or non-normalized mRNA levels in different samples.

The gene expression profile data can be in the form of a table, such as an Excel table. The data can be alone, or it can be part of a larger database, e.g., comprising other expression profiles. For example, the expression profile data of the invention can be part of a public database. The computer readable form can be in a computer. In another embodiment, the invention provides a computer displaying the gene expression profile data.

In one embodiment, the invention provides a method for determining the similarity between the level of expression of one or more “BREAST CANCER GENES” in a first cell, e.g., a cell of a subject, and that in a second cell, comprising obtaining the level of expression of one or more “BREAST CANCER GENES” in a first cell and entering these values into a computer comprising a database including records comprising values corresponding to levels of expression of one or more “BREAST CANCER GENES” in a second cell, and processor instructions, e.g., a user interface, capable of receiving a selection of one or more values for comparison purposes with data that is stored in the computer. The computer may further comprise a means for converting the comparison data into a diagram or chart or other type of output.

In another embodiment, values representing expression levels of “BREAST CANCER GENES” are entered into a computer system, comprising one or more databases with reference expression levels obtained from more than one cell. For example, the computer comprises expression data of diseased and normal cells. Instructions are provided to the computer, and the computer is capable of comparing the data entered with the data in the computer to determine whether the data entered is more similar to that of a normal cell or of a diseased cell.

In another embodiment, the computer comprises values of expression levels in cells of subjects at different stages of breast cancer, and the computer is capable of comparing expression data entered into the computer with the data stored, and produce results indicating to which of the expression profiles in the computer, the one entered is most similar, such as to determine the stage of breast cancer in the subject.

In yet another embodiment, the reference expression profiles in the computer are expression profiles from cells of breast cancer of one or more subjects, which cells are treated in vivo or in vitro with a drug used for therapy of breast cancer. Upon entering of expression data of a cell of a subject treated in vitro or in vivo with the drug, the computer is instructed to compare the data entered to the data in the computer, and to provide results indicating whether the expression data input into the computer are more similar to those of a cell of a subject that is responsive to the drug or more similar to those of a cell of a subject that is not responsive to the drug. Thus, the results indicate whether the subject is likely to respond to the treatment with the drug or unlikely to respond to it.

In one embodiment, the invention provides a system that comprises a means for receiving gene expression data for one or a plurality of genes; a means for comparing the gene expression data from each of said one or plurality of genes to a common reference frame; and a means for presenting the results of the comparison. This system may further comprise a means for clustering the data.

In addition we challenged a classical PCA algorithm with the identification of the major components separating the samples and the two therapeutic outcomes.

In another embodiment, the invention provides a computer program for analyzing gene expression data comprising (i) a computer code that receives as input gene expression data for a plurality of genes and (ii) a computer code that compares said gene expression data from each of said plurality of genes to a common reference frame.

The invention also provides a machine-readable or computer-readable medium including program instructions for performing the following steps: (i) comparing a plurality of values corresponding to expression levels of one or more genes characteristic of breast cancer in a query cell with a database including records comprising reference expression or expression profile data of one or more reference cells and an annotation of the type of cell; and (ii) indicating to which cell the query cell is most similar based on similarities of expression profiles. The reference cells can be cells from subjects at different stages of breast cancer. The reference cells can also be cells from subjects responding or not responding to a particular drug treatment and optionally incubated in vitro or in vivo with the drug.

The reference cells may also be cells from subjects responding or not responding to several different treatments, and the computer system indicates a preferred treatment for the subject. Accordingly, the invention provides a method for selecting a therapy for a patient having breast cancer, the method comprising: (i) providing the level of expression of one or more genes characteristic of breast cancer in a diseased cell of the patient; (ii) providing a plurality of reference profiles, each associated with a therapy, wherein the subject expression profile and each reference profile has a plurality of values, each value representing the level of expression of a gene characteristic of breast cancer; and (iii) selecting the reference profile most similar to the subject expression profile, to thereby select a therapy for said patient. In a preferred embodiment step (iii) is performed by a computer. The most similar reference profile may be selected by weighing a comparison value of the plurality using a weight value associated with the corresponding expression data.

The relative abundance of an mRNA in two biological samples can be scored as a perturbation and its magnitude determined (i.e., the abundance is different in the two sources of mRNA tested), or as not perturbed (i.e., the relative abundance is the same). In various embodiments, a difference between the two sources of RNA of at least a factor of about 25% (RNA from one source is 25% more abundant in one source than the other source), more usually about 50%, even more often by a factor of about 2 (twice as abundant), 3 (three times as abundant) or 5 (five times as abundant) is scored as a perturbation. Perturbations can be used by a computer for calculating and expression comparisons.

Preferably, in addition to identifying a perturbation as positive or negative, it is advantageous to determine the magnitude of the perturbation. This can be carried out, as noted above, by calculating the ratio of the emission of the two fluorophores used for differential labeling, or by analogous methods that will be readily apparent to those of skill in the art.

The computer readable medium may further: comprise a pointer to a descriptor of a stage of breast cancer or to a treatment for breast cancer.

In operation, the means for receiving gene expression data, the means for comparing the gene expression data, the means for presenting, the means for normalizing, and the means for clustering within the context of the systems of the present invention can involve a programmed computer with the respective functionalities described herein, implemented in hardware or hardware and software; a logic circuit or other component of a programmed computer that performs the operations specifically identified herein, dictated by a computer program; or a computer memory encoded with executable instructions representing a computer program that can cause a computer to function in the particular fashion described herein.

Those skilled in the art will understand that the systems and methods of the present invention may be applied to a variety of systems, including IBM-compatible personal computers running MS-DOS or Microsoft Windows.

The computer may have internal components linked to external components. The internal components may include a processor element interconnected with a main memory. The computer system can be an Intel Pentium®-based processor of 200 MHz or greater clock rate and with 32 MB or more of main memory. The external component may comprise a mass storage, which can be one or more hard disks (which are typically packaged together with the processor and memory).

Such hard disks are typically of 1 GB or greater storage capacity. Other external components include a user interface device, which can be a monitor, together with an inputing device, which can be a “mouse”, or other graphic input devices, and/or a keyboard. A printing device can also be attached to the computer.

Typically, the computer system is also linked to a network link, which can be part of an Ethernet link to other local computer systems, remote computer systems, or wide area communication networks, such as the Internet. This network link allows the computer system to share data and processing tasks with other computer systems.

Loaded into memory during operation of this system are several software components, which are both standard in the art and special to the instant invention. These software components collectively cause the computer system to function according to the methods of this invention. These software components are typically stored on a mass storage. A software component represents the operating system, which is responsible for managing the computer system and its network interconnections. This operating system can be, for example, of the Microsoft Windows' family, such as Windows 95, Windows 98, or Windows NT. A software component represents common languages and functions conveniently present on this system to assist programs implementing the methods specific to this invention. Many high or low level computer languages can be used to program the analytic methods of this invention. Instructions can be interpreted during run-time or compiled. Preferred languages include C/C++, and JAVA®. Most preferably, the methods of this invention are programmed in mathematical software packages which allow symbolic entry of equations and high-level specification of processing, including algorithms to be used, thereby freeing a user of the need to procedurally program individual equations or algorithms. Such packages include Matlab from Mathworks (Natick, Mass.), Mathematica from Wolfram Research (Champaign, Ill.), or S-Plus from Math Soft (Cambridge, Mass.). Accordingly, a software component represents the analytic methods of this invention as programmed in a procedural language or symbolic package. In a preferred embodiment, the computer system also contains a database comprising values representing levels of expression of one or more genes characteristic of breast cancer. The database may contain one or more expression profiles of genes characteristic of breast cancer in different cells.

In an exemplary implementation, to practice the methods of the present invention, a user first loads expression profile data into the computer system. These data can be directly entered by the user from a monitor and keyboard, or from other computer systems linked by a network connection, or on removable storage media such as a CD-ROM or floppy disk or through the network. Next the user causes execution of expression profile analysis software which performs the steps of comparing and, e.g., clustering co-varying genes into groups of genes.

In another exemplary implementation, expression profiles are compared using a method described in U.S. Pat. No. 6,203,987. A user first loads expression profile data into the computer system. Geneset profile definitions are loaded into the memory from the storage media or from a remote computer, preferably from a dynamic geneset database system, through the network. Next the user causes execution of projection software which performs the steps of converting expression profile to projected expression profiles. The projected expression profiles are then displayed.

In yet another exemplary implementation, a user first leads a projected profile into the memory. The user then causes the loading of a reference profile into the memory. Next, the user causes the execution of comparison software which performs the steps of objectively comparing the profiles.

### In Situ Hybridization

In one aspect, the method comprises in situ hybridization with a probe derived from a given marker polynucleotide, which sequence is selected from any of the polynucleotide sequences of the genes listed in Table 1 or a sequence complementary thereto. The method comprises contacting the labeled hybridization probe with a sample of a given type of tissue from a patient potentially having malignant neoplasia and breast cancer in particular as well as normal tissue from a person with no malignant neoplasia, and determining whether the probe labels tissue of the patient to a degree significantly different (e.g., by at least a factor of two, or at least a factor of five, or at least a factor of twenty, or at least a factor of fifty) than the degree to which normal tissue is labelled. In situ hybridization may be performed either to DNA in the nucleus of said cell in tissues or to the mRNA in the cytoplasm to stain for transcriptional activity.

### Polypeptide Detection

The subject invention further provides a method of determining whether a cell sample obtained from a subject possesses an abnormal amount of marker polypeptide which comprises (a) obtaining a cell sample from the subject, (b) quantitatively determining the amount of the marker polypeptide in the sample so obtained, and (c) comparing the amount of the marker polypeptide so determined with a known standard, so as to thereby determine whether the cell sample obtained from the subject possesses an abnormal amount of the marker polypeptide. Such marker polypeptides may be detected by immunohistochemical assays, dot-blot assays, ELISA and the like.

### Antibodies

Any type of antibody known in the art can be generated to bind specifically to an epitope of a “BREAST CANCER GENE” polypeptide. An antibody as used herein includes intact immuno-globulin molecules, as well as fragments thereof, such as Fab, F(ab)₂, and Fv, which are capable of binding an epitope of a “BREAST CANCER GENE” polypeptide. Typically, at least 6, 8, 10, or 12 contiguous amino acids are required to form an epitope. However, epitopes which involve non-contiguous amino acids may require more, e.g., at least 15, 25, or 50 amino acids.

An antibody which specifically binds to an epitope of a “BREAST CANCER GENE” polypeptide can be used therapeutically, as well as in immunochemical assays, such as Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody which specifically binds to the immunogen.

Typically, an antibody which specifically binds to a “BREAST CANCER GENE” polypeptide provides a detection signal at least 5-, 10-, or 20-fold higher than a detection signal provided with other proteins when used in an immunochemical assay. Preferably, antibodies which specifically bind to “BREAST CANCER GENE” polypeptides do not detect other proteins in immunochemical assays and can immunoprecipitate a “BREAST CANCER GENE” polypeptide from solution.

“BREAST CANCER GENE” polypeptides can be used to immunize a mammal, such as a mouse, rat, rabbit, guinea pig, monkey, or human, to produce polyclonal antibodies. If desired, a “BREAST CANCER GENE” polypeptide can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a “BREAST CANCER GENE” polypeptide can be prepared using any technique which provides for the production of antibody molecules by continuous cell lines in culture. These techniques include, but are not limited to, the hybridoma technique, the human B cell hybridoma technique, and the EBV hybridoma technique [Kohler et al., 1985, (13)].

In addition, techniques developed for the production of chimeric antibodies, the splicing of mouse antibody genes to human antibody genes to obtain a molecule with appropriate antigen specificity and biological activity, can be used [Takeda et al., 1985, (14)]. Monoclonal and other antibodies also can be humanized to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions. Alternatively, humanized antibodies can be produced using recombinant methods, as described in GB2188638B. Antibodies which specifically bind to a “BREAST CANCER GENE” polypeptide can contain antigen binding sites which are either partially or fully humanized, as disclosed in U.S. Pat. No. 5,565,332.

Alternatively, techniques described for the production of single chain antibodies can be adapted using methods known in the art to produce single chain antibodies which specifically bind to “BREAST CANCER GENE” polypeptides. Antibodies with related specificity, but of distinct idiotypic composition, can be generated by chain shuffling from random combinatorial immunoglobulin libraries [Burton, 1991, (15)].

Single-chain antibodies also can be constructed using a DNA amplification method, such as PCR, using hybridoma cDNA as a template [Thirion et al., 1996, (16)]. Single-chain antibodies can be mono- or bispecific, and can be bivalent or tetravalent. Construction of tetravalent, bispecific single-chain antibodies is taught, for example, in Coloma & Morrison, (17). Construction of bivalent, bispecific single-chain antibodies is taught in Mallender & Voss, (18).

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology [Verhaar et al., 1995, (19)].

Antibodies which specifically bind to “BREAST CANCER GENE” polypeptides also can be produced by inducing in vivo production in the lymphocyte population or by screening immunoglobulin libraries or panels of highly specific binding reagents as disclosed in the literature [Orlandi et al., 1989, (20)].

Other types of antibodies can be constructed and used therapeutically in methods of the invention. For example, chimeric antibodies can be constructed as disclosed in WO 93/03151. Binding proteins which are derived from immunoglobulins and which are multivalent and multispecific, such as the antibodies described in WO 94/13804, also can be prepared.

Antibodies according to the invention can be purified by methods well known in the art. For example, antibodies can be affinity purified by passage over a column to which a “BREAST CANCER GENE” polypeptide is bound. The bound antibodies can then be eluted from the column using a buffer with a high salt concentration.

Immunoassays are commonly used to quantify the levels of proteins in cell samples, and many other immunoassay techniques are known in the art. The invention is not limited to a particular assay procedure, and therefore is intended to include both homogeneous and heterogeneous procedures. Exemplary immunoassays which can be conducted according to the invention include fluorescence polarisation immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art.

Other methods to quantify the level of a particular protein, or a protein fragment, or modified protein in a particular sample are based on flow-cytometric methods. Flow cytometry allows the identification of proteins on the cell surface as well as of intracellular proteins using fluorochrome labeled, protein specific antibodies or non-labeled antibodies in combination with fluorochrome labeled secondary antibodies. General techniques to be used in performing flow cytometric assays noted above are known to those of ordinary skill in the art. A special method based on the same principles is the microsphere-based flow cytometric. Microsphere beads are labeled with precise quantities of fluorescent dye and particular antibodies. Such techniques are provided by Luminex Inc. WO 97/14028. In another embodiment the level of a particular protein or a protein fragment, or modified protein in a particular sample may be determined by 2D gel-electrophoresis and/or mass spectrometry. Determination of protein nature, sequence, molecular mass as well charge can be achieved in one detection step. Mass spectrometry can be performed with methods known to those with skills in the art as MALDI, TOF, or combinations of these.

In another embodiment, the level of the encoded product, i.e., the product encoded by any of the polynucleotide sequences of the genes listed in Table 1 or a sequence complementary thereto, in a biological fluid (e.g., blood or urine) of a patient may be determined as a way of monitoring the level of expression of the marker polynucleotide sequence in cells of that patient. Such a method would include the steps of obtaining a sample of a biological fluid from the patient, contacting the sample (or proteins from the sample) with an antibody specific for a encoded marker polypeptide, and determining the amount of immune complex formation by the antibody, with the amount of immune complex formation being indicative of the level of the marker encoded product in the sample. This determination is particularly instructive when compared to the amount of immune complex formation by the same antibody in a control sample taken from a normal individual or in one or more samples previously or subsequently obtained from the same person.

In another embodiment, the method can be used to determine the amount of marker polypeptide present in a cell, which in turn can be correlated with progression of the disorder, e.g., plaque formation. The level of the marker polypeptide can be used predictively to evaluate whether a sample of cells contains cells which are, or are predisposed towards becoming, plaque associated cells. The observation of marker polypeptide level can be utilized in decisions regarding, e.g., the use of more stringent therapies.

As set out above, one aspect of the present invention relates to diagnostic assays for determining, in the context of cells isolated from a patient, if the level of a marker polypeptide is significantly reduced in the sample cells. The term “significantly reduced” refers to a cell phenotype wherein the cell possesses a reduced cellular amount of the marker polypeptide relative to a normal cell of similar tissue origin. For example, a cell may have less than about 50%, 25%, 10%, or 5% of the marker polypeptide that a normal control cell. In particular, the assay evaluates the level of marker polypeptide in the test cells, and, preferably, compares the measured level with marker polypeptide detected in at least one control cell, e.g., a normal cell and/or a transformed cell of known phenotype.

Of particular importance to the subject invention is the ability to quantify the level of marker polypeptide as determined by the number of cells associated with a normal or abnormal marker polypeptide level. The number of cells with a particular marker polypeptide phenotype may then be correlated with patient prognosis. In one embodiment of the invention, the marker polypeptide phenotype of the lesion is determined as a percentage of cells in a biopsy which are found to have abnormally high/low levels of the marker polypeptide. Such expression may be detected by immunohistochemical assays, dot-blot assays, ELISA and the like.

### Immunohistochemistry

Where tissue samples are employed, immunohistochemical staining may be used to determine the number of cells having the marker polypeptide phenotype. For such staining, a multiblock of tissue is taken from the biopsy or other tissue sample and subjected to proteolytic hydrolysis, employing such agents as protease K or pepsin. In certain embodiments, it may be desirable to isolate a nuclear fraction from the sample cells and detect the level of the marker polypeptide in the nuclear fraction.

The tissues samples are fixed by treatment with a reagent such as formalin, glutaraldehyde, methanol, or the like. The samples are then incubated with an antibody, preferably a monoclonal antibody, with binding specificity for the marker polypeptides. This antibody may be conjugated to a Label for subsequent detection of binding. samples are incubated for a time Sufficient for formation of the immunocomplexes. Binding of the antibody is then detected by virtue of a Label conjugated to this antibody. Where the antibody is unlabelled, a second labeled antibody may be employed, e.g., which is specific for the isotype of the anti-marker polypeptide antibody. Examples of labels which may be employed include radionuclides, fluorescence, chemoluminescence, and enzymes.

Where enzymes are employed, the Substrate for the enzyme may be added to the samples to provide a colored or fluorescent product. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art.

In one embodiment, the assay is performed as a dot blot assay. The dot blot assay finds particular application where tissue samples are employed as it allows determination of the average amount of the marker polypeptide associated with a Single cell by correlating the amount of marker polypeptide in a cell-free extract produced from a predetermined number of cells.

In yet another embodiment, the invention contemplates using a panel of antibodies which are generated against the marker polypeptides of this invention, which polypeptides are encoded by any of the polynucleotide sequences of the genes from Table 1. Such a panel of antibodies may be used as a reliable diagnostic probe for breast cancer. The assay of the present invention comprises contacting a biopsy sample containing cells, e.g., macrophages, with a panel of antibodies to one or more of the encoded products to determine the presence or absence of the marker polypeptides.

The diagnostic methods of the subject invention may also be employed as follow-up to treatment, e.g., quantification of the level of marker polypeptides may be indicative of the effectiveness of current or previously employed therapies for malignant neoplasia and breast cancer in particular as well as the effect of these therapies upon patient prognosis.

The diagnostic assays described above can be adapted to be used as prognostic assays, as well. Such an application takes advantage of the sensitivity of the assays of the Invention to events which take place at characteristic stages in the progression of plaque generation in case of malignant neoplasia. For example, a given marker gene may be up- or down-regulated at a very early stage, perhaps before the cell is developing into a foam cell, while another marker gene may be characteristically up or down regulated only at a much later stage. Such a method could involve the steps of contacting the mRNA of a test cell with a polynucleotide probe derived from a given marker polynucleotide which is expressed at different characteristic levels in breast cancer tissue cells at different stages of malignant neoplasia progression, and determining the approximate amount of hybridization of the probe to the mRNA of the cell, such amount being an indication of the level of expression of the gene in the cell, and thus an indication of the stage of disease progression of the cell; alternatively, the assay can be carried out with an antibody specific for the gene product of the given marker polynucleotide, contacted with the proteins of the test cell. A battery of such tests will disclose not only the existence of a certain neoplastic lesion, but also will allow the clinician to select the mode of treatment most appropriate for the disease, and to predict the likelihood of success of that treatment.

The methods of the invention can also be used to follow the clinical course of a given breast cancer predisposition. For example, the assay of the Invention can be applied to a blood sample from a patient; following treatment of the patient for BREAST CANCER, another blood sample is taken and the test repeated. Successful treatment will result in removal of demonstrate differential expression, characteristic of the breast cancer tissue cells, perhaps approaching or even surpassing normal levels. Modulation of Gene Expression

In another embodiment, test compounds which increase or decrease “BREAST CANCER GENE” expression are identified. A “BREAST CANCER GENE” polynucleotide is contacted with a test compound in an appropriate expression test system as described below or in a cell system, and the expression of an RNA or polypeptide product of the “BREAST CANCER GENE” polynucleotide is determined. The level of expression of appropriate mRNA or polypeptide in the presence of the test compound is compared to the level of expression of mRNA or polypeptide in the absence of the test compound. The test compound can then be identified as a modulator of expression based on this comparison. For example, when expression of mRNA or polypeptide is greater in the presence of the test compound than in its absence, the test compound is identified as a stimulator or enhancer of the mRNA or polypeptide expression. Alternatively, when expression of the mRNA or polypeptide is less in the presence of the test compound than in its absence, the test compound is identified as an inhibitor of the mRNA or polypeptide expression.

The level of “BREAST CANCER GENE” mRNA or polypeptide expression in the cells can be determined by methods well known in the art for detecting mRNA or polypeptide. Either qualitative or quantitative methods can be used. The presence of polypeptide products of a “BREAST CANCER GENE” polynucleotide can be determined, for example, using a variety of techniques known in the art, including immunochemical methods such as radioimmunoassay, Western blotting, and immunohistochemistry. Alternatively, polypeptide synthesis can be determined in vivo, in a cell culture, or in an in vitro translation system by detecting incorporation of labeled amino acids into a “BREAST CANCER GENE” polypeptide.

Such screening can be carried out either in a cell-free assay system or in an intact cell. Any cell which expresses a “BREAST CANCER GENE” polynucleotide can be used in a cell-based assay system. A “BREAST CANCER GENE” polynucleotide can be naturally occurring in the cell or can be introduced using techniques such as those described above. Either a primary culture or an established cell line, such as CHO or human embryonic kidney 293 cells, can be used.

One strategy for identifying genes that are involved in breast cancer is to detect genes that are expressed differentially under conditions associated with the disease versus non-disease or in the context of therapy response conditions. The sub-sections below describe a number of experimental systems which can be used to detect such differentially expressed genes. In general, these experimental systems include at least one experimental condition in which subjects or samples are treated in a manner associated with breast cancer, in addition to at least one experimental control condition lacking such disease associated treatment or does not respond to such treatment. Differentially expressed genes are detected, as described below, by comparing the pattern of gene expression between the experimental and control conditions.

Once a particular gene has been identified through the use of one such experiment, its expression pattern may be further characterized by studying its expression in a different experiment and the findings may be validated by an independent technique. Such use of multiple experiments may be useful in distinguishing the roles and relative importance of particular genes in breast cancer and the treatment thereof. A combined approach, comparing gene expression pattern in cells derived from breast cancer patients to those of in vitro cell culture models can give substantial hints on the pathways involved in development and/or progression of breast cancer. It can also elucidate the role of such genes in the development of resistance or insensitivity to certain therapeutic agents (e.g. chemotherapeutic drugs).

Among the experiments which may be utilized for the identification of differentially expressed genes involved in malignant neoplasia and breast cancer in particular, are experiments designed to analyze those genes which are involved in signal transduction. Such experiments may serve to identify genes involved in the proliferation of cells.

Below are methods described for the identification of genes which are involved in breast cancer. Such represent genes which are differentially expressed in breast cancer conditions relative to their expression in normal, or non-breast cancer conditions or upon experimental manipulation based on clinical observations. Such differentially expressed genes represent “target” and/or “marker” genes. Methods for the further characterization of such differentially expressed genes, and for their identification as target and/or marker genes, are presented below.

Alternatively, a differentially expressed gene may have its expression modulated, i.e., quantitatively increased or decreased, in normal versus breast cancer states, or under control versus experimental conditions. The degree to which expression differs in normal versus breast cancer or control versus experimental states need only be large enough to be visualized via standard characterization techniques, such as, for example, the differential display technique described below. Other such standard characterization techniques by which expression differences may be visualized include but are not limited to quantitative RT-PCR and Northern analyses, which are well known to those of skill in the art.

In Addition to the experiments described above the following describes algorithms and statistical analyses which can be utilized for data evaluation and for the classification as well as response prediction for a sofar not classified biological sample in the context of control samples. Predictive algorithms and equations described below have already shown their power to subdivide individual cancers.

### EXAMPLE 1

### Expression Profiling Utilizing Quantitative Kinetic RT-PCR

For a detailed analysis of gene expression by quantitative PCR methods, one will utilize primers flanking the genomic region of interest and a fluorescent labeled probe hybridizing in-between. Using the PRISM 7700 Sequence Detection System of PE Applied Biosystems (Perkin Elmer, Foster City, Calif., USA) with the technique of a fluorogenic probe, consisting of an oligonucleotide labeled with both a fluorescent reporter dye and a quencher dye, one can perform such a expression measurement. Amplification of the probe-specific product causes cleavage of the probe, generating an increase in reporter fluorescence. Primers and probes were selected using the Primer Express software and localized mostly in the 3′ region of the coding sequence or in the 3′ untranslated region. Primer design and selection of an appropriate target region is well known to those with skills in the art. Predefined primer and probes for the genes listed in Table 1 can also be obtained from suppiers e.g. PE Applied Biosystems. All primer pairs were checked for specificity by conventional PCR reactions and gel electrophoresis. To standardize the amount of sample RNA, GAPDH was selected as a reference, since it was not differentially regulated in the samples analyzed. To performed such an expression analysis of genes within a biological samples the respective primer/probes are prepared by mixing 25 μl of the 100 μM stock solution “Upper Primer”, 25 μl of the 100 μM stock solution “Lower Primer” with 12.5 μl of the 100 μM stock solution TaqMan-probe (FAM/Tamra) and adjusted to 500 μl with aqua dest (Primer/probe-mix). For each reaction 1.25 μl cDNA of the patient samples were mixed with 8.75 μl nuclease-free water and added to one well of a 96 Well-Optical Reaction Plate (Applied Biosystems Part No. 4306737). 1.5 μl of the Primer/Probe-mix described above, 12.5 μl Taq Man Universal-PCR-mix (2×) (Applied Biosystems Part No. 4318157) and 1 μl Water are then added. The 96 well plates are closed with 8 Caps/Strips (Applied Biosystems Part Number 4323032) and centrifuged for 3 minutes. Measurements of the PCR reaction are done according to the instructions of the manufacturer with a TaqMan 7700 from Applied Biosystems (No. 20114) under appropriate conditions (2 min. 50° C., 10 min. 95° C., 0.15 min. 95° C., 1 min. 60° C.; 40 cycles). Prior to the measurement of so far unclassified biological samples control experiments will e.g. cell lines, healthy control samples, samples of defined therapy response could be used for standardization of the experimental conditions.

TaqMan validation experiments were performed showing that the efficiencies of the target and the control amplifications are approximately equal which is a prerequisite for the relative quantification of gene expression by the comparative ΔΔCT method, known to those with skills in the art. Herefor the SoftwareSDS 2.0 from Applied Biosystems can be used according to the respective instructions. CT-values are then further analyzed with appropriate software (Microsoft Excel™) of statistical software packages (SAS).

As well as the technology described above, provided by Perkin Elmer, one may use other technique implementations like Lightcycler™ from Roche Inc. or iCycler from Stratagene Inc. capable of real time detection of an RT-PCR reaction.

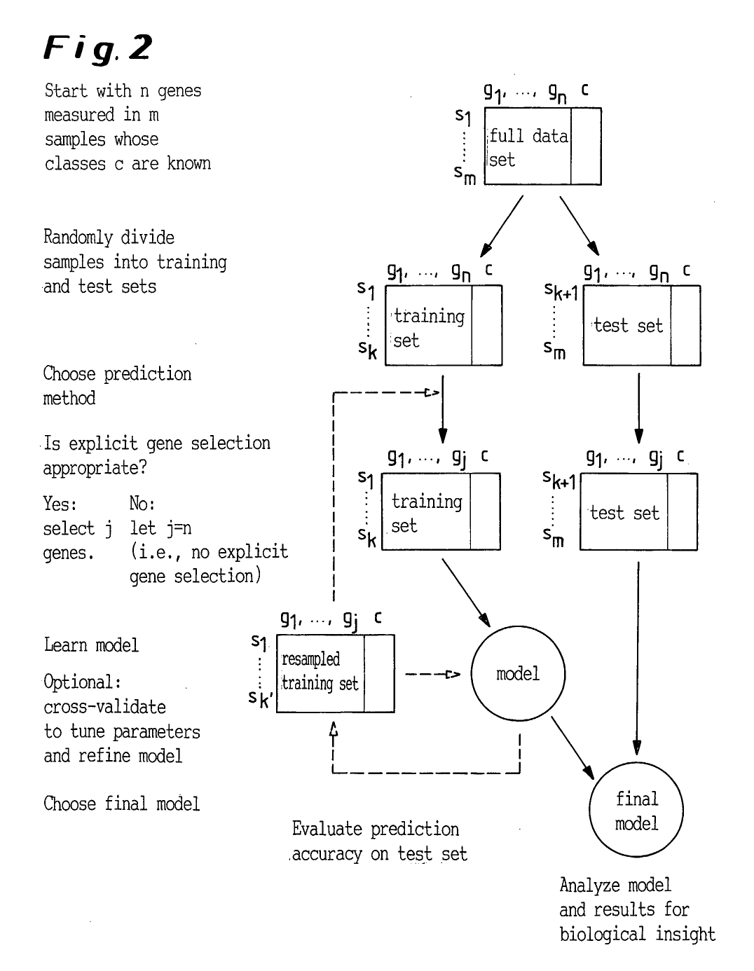

TABLE 1
84 Genes differentially expressed and capable of predicting therapeutic success.

| SEQ | GENE | | REF. | LOCUS | UNIGENE | |
|---|---|---|---|---|---|---|
| NO | SYMBOL | GENE DESCRIPTION | SEQUENCES | LINK ID | ID | OMIM |
| 1 | ADAM17 | HSU69611 TNF-alpha converting enzyme a | NM_003183 | 6868 | 64311 | 603639 |
| | | disintegrin and metalloproteinase domain | | | | |
| | | 17 (tumor necrosis factor alpha converting | | | | |
| | | enzyme) transmembrane | | | | |
| | | metalloproteinase/disintegrin; adamalysin; | | | | |
| | | TACEA TNF-alpha converting enzyme | | | | |
| 2 | ANKT | yr78b09.s1 uncharacterized bone marrow | NM_016359 | 51203 | 283649 | — |
| | | protein BM037 clone HQ0310 PRO0310p1 | | | | |
| 3 | APRT | APRT geneadenine | NM_000485 | 353 | 28914 | 102600 |
| | | phosphoribosyltransferase adenine | | | | |
| | | phosphoribosyltransferase adenine | | | | |
| | | phosphoribosyltransferase; aprt gene | | | | |
| | | adenine phosphoribosyltransferase (aprt) | | | | |
| 4 | ASK | activator of S phase Kinase activator of S | NM_006716 | 10926 | 152759 | 604281 |
| | | phase kinase activator of S phase Kinase | | | | |
| | | ASK encodes a regulatory subunit for | | | | |
| | | huCdc7, the human homologue of budding | | | | |
| | | yeast Cdc7 kinase.; | | | | |
| 5 | BIGM103 | DKFZp564A132 (from clone | NM_022154 | 64116 | 284205 | — |
| | | DKFZp564A132) for BCG induced integral | | | | |
| | | membrane protein BIGMo-103d Homo | | | | |
| | | sapiens mRNA; cDNA DKFZp564A132 | | | | |
| | | (from clone DKFZp564A132) up-regulated | | | | |
| | | by BCG-CWS | | | | |
| 6 | BRAG | KIAA0598 protein B cell RAG associated | NM_014863 | 51363 | 6079 | — |
| | | protein KIAA0598 protein KIAA0598 gene | | | | |
| | | product | | | | |
| 7 | BTBD3 | KIAA0952 protein KIAA0952 protein | NM_014962 | 22903 | 7935 | — |
| | | KIAA0952 protein BTB (POZ) domain | | | | |
| | | containing 3 | | | | |
| 8 | BTG3 | ANA BTG family member 3 ANA BTG | NM_006806 | 10950 | 77311 | 605674 |
| | | family, member 3 | | | | |
| 9 | CCNB1 | HUMCYCB cyclin B3″ end cyclin B1 cyclin B | NM_031966 | 891 | 23960 | 123836 |
| 10 | CDC25B | S78187 CDC25Hu2 cdc25+ homolog | NM_004358 | 994 | 153752 | 116949 |
| | | [3118 nt] cell division cycle 25B (CDC25B) | | | | |
| | | transcript variant cell division cycle 25B | | | | |
| | | p63; This sequence comes from FIG. 2 cell | | | | |
| | | division cycle 25B | | | | |
| 11 | CDKN2A | HSU26727 p16INK4 MTS1 cyclin- | NM_000077 | 1029 | 1174 | 600160 |
| | | dependent kinase inhibitor 2A (melanoma | | | | |
| | | p16 inhibits CDK4) a frameshift between | | | | |
| | | exon 1 (0.18) and exon 2 changed the ORF | | | | |
| | | of p16INK4 gene. cyclin-dependent kinase | | | | |
| | | inhibitor 2A (melanoma, p16, inhibits | | | | |
| | | CDK4) | | | | |
| 12 | CDT1 | clone 24767 sequence clone | NM_030928 | 81620 | 122908 | 605525 |
| | | IMAGE: 3048353 ds Homo sapiens clone | | | | |
| | | 24767 mRNA sequence FLI_CDNA DNA | | | | |
| | | replication factor | | | | |
| 13 | CEACAM6 | nonspecific crossreacting antigen | NM_002483 | 4680 | 73848 | 163980 |
| | | carcinoembryonic antigen-related cell | | | | |
| | | adhesion molecule 6 (non-specific cross | | | | |
| | | reacting antigen) clone MGC: 104 | | | | |
| | | nonspecific cross-reacting antigen ORF1 | | | | |
| | | non-specific cross reacting antigen | | | | |
| 14 | CENPF | mitosin centromere protein F (350400 kD | NM_016343 | 1063 | 77204 | 600236 |
| | | mitosin) centromere protein F (350/400 kD | | | | |
| | | mitosin) 350 kDa nuclear phosphoprotein | | | | |
| | | centromere protein F (350/400 kD, mitosin) | | | | |
| 15 | CTSL2 | cathepsin V cathepsin U (CTSU) d | NM_001333 | 1515 | 87417 | 603308 |
| | | cathepsin L2 | | | | |
| 16 | CXADR | 46 kDa coxsackievirus and adenovirus | NM_001338 | 1525 | 79187 | 602621 |
| | | receptor (CAR) protein coxsackie virus and | | | | |
| | | adenovirus receptor 46 kDa receptor | | | | |
| | | protein; coxsackie and adenovirus receptor | | | | |
| | | protein coxsackie virus and adenovirus | | | | |
| | | receptor | | | | |
| 17 | DDA3 | wq62b06.x1 Similar to differential display | NM_001826 | 1163 | 77550 | 116900 |
| | | and activated by p53 clone MGC: 17 | | | | |
| | | prothymosin alpha (gene sequence 28) | | | | |
| | | EST CDC28 protein kinase 1 | | | | |
| 18 | DSG3 | 130-kD pemphigus vulgaris antigen | NM_001944 | 1830 | 1925 | 169615 |
| | | desmoglein 3 (pemphigus vulgaris antigen) | | | | |
| | | autoantibody; cadherin; pemphigus vulgaris | | | | |
| | | antigen 130-kD pemphigus vulgaris antigen | | | | |
| 19 | DUSP9 | MAP kinase phosphatase 4 dual specificity | NM_001395 | 1852 | 144879 | 300134 |
| | | phosphatase 9 | | | | |
| 20 | E2-EPF | HUME2EPI ubiquitin carrier protein (E2- | NM_014501 | 27338 | 174070 | — |
| | | EPF) ubiquitin carrier protein | | | | |
| 21 | ENO1 | HUMCMYCQ c-myc binding protein (MBP- | NM_001428 | 2023 | 254105 | 172430 |
| | | 1) alpha enolase like 1 (ENO1L1) d MYC | | | | |
| | | promoter-binding protein 1 c-myc binding | | | | |
| | | protein | | | | |
| 22 | FLJ10079 | yj44b02.r1 hypothetical protein FLJ10079 | NM_017990 | 55066 | 261215 | — |
| 23 | FLJ10156 | no87e07.s1 hypothetical protein FLJ10491 | NM_019013 | 54478 | 86211 | — |
| | | clone MGC: 9 hypothetical protein EST | | | | |
| 24 | FLJ12949 | zo29h06.s1 hypothetical protein FLJ12949 | NM_023008 | 65095 | 184519 | — |
| | | Homo sapiens cDNA FLJ12949 fis clone | | | | |
| | | NT2RP2005336 weakly similar to | | | | |
| | | TRICHOHYALIN EST | | | | |
| 25 | FLJ20354 | wf65c09.x1 hypothetical protein FLJ20354 | NM_017779 | 55635 | 133260 | — |
| | | EST | | | | |
| 26 | FLJ20364 | zq56a12.s1 arsenite related gene 1 d | NM_017785 | 54908 | 32471 | — |
| | | hypothetical protein FLJ20364 EST | | | | |
| 27 | FLJ21313 | tc72c09.x1 hypothetical protein FLJ21313 | NM_023927 | 65983 | 235445 | — |
| | | Homo sapiens cDNA: FLJ21313 fis clone | | | | |
| | | COL02176 | | | | |
| 28 | FLJ22029 | yc79e04.s1 hypothetical protein FLJ22029 | NM_024949 | 80014 | 285243 | — |
| | | Homo sapiens cDNA: FLJ22029 fis clone | | | | |
| | | HEP08661 EST | | | | |
| 29 | FOLR1 | HSU20391 folate receptor (FOLR1) gene | NM_000802 | 2348 | 73769 | 136430 |
| | | folate receptor 1 (adult) (FOLR1) transcript | | | | |
| | | variant folate receptor 1 (adult) | | | | |
| 30 | GATM | L-arginine-glycine amidinotransferase | NM_001482 | 2628 | 75335 | 602360 |
| | | [kidney carcinoma cells 2330 nt] glycine | | | | |
| | | amidinotransferase (L-arginine:glycine | | | | |
| | | amidinotransferase) This sequence comes | | | | |
| | | from FIG. 4 glycine amidinotransferase (L- | | | | |
| | | arginine:glycine amidinotransferase) | | | | |
| 31 | GLUL | rearranged glutamine synthase glutamate- | NM_002065 | 2752 | 170171 | 138290 |
| | | ammonia ligase (glutamine synthase) | | | | |
| 32 | GPR56 | TM7XN1 protein G protein-coupled | NM_005682 | 9289 | 6527 | 604110 |
| | | receptor 56 EGF-TM7-like protein; TM7XN1 | | | | |
| | | protein EGF-TM7 like protein G protein- | | | | |
| | | coupled receptor 56 | | | | |
| 33 | GTSE1 | DNA sequence from Fosmid 27C3 on | NM_016426 | 51512 | 122552 | 607477 |
| | | chromosome 22q11.2-qter. Contains two | | | | |
| | | possibly alternatively spliced unknown | | | | |
| | | genes one with homology to a worm | | | | |
| | | protein. Contains ESTs G-2 and S-phase | | | | |
| | | expressed 1 hypothetical protein FLJ10140 | | | | |
| 34 | HEMK | zd97g03.s1 HEMK homolog (HEMK) d | NM_016173 | 51409 | 46907 | — |
| | | HEMK homolog 7 kb EST | | | | |
| 35 | HMMR | intracellular hyaluronic acid binding protein | NM_012484 | 3161 | 72550 | 600936 |
| | | (IHABP) hyaluronan-mediated motility | | | | |
| | | receptor (RHAMM) (HMMR) transcript | | | | |
| | | variant hyaluronan-mediated motility | | | | |
| | | receptor (RHAMM) expressed in breast | | | | |
| | | cancer cells hyaluronan-mediated motility | | | | |
| | | receptor (RHAMM) | | | | |
| 36 | KARS | Lysyl tRNA Synthetase lysyl-tRNA | NM_005548 | 3735 | 3100 | 601421 |
| | | synthetase Lysyl tRNA Synthetase | | | | |
| 37 | KCNK5 | wt09d07.x1 potassium channel subfamily K | NM_003740 | 8645 | 127007 | 603493 |
| | | member 5 (TASK-2) | | | | |
| 38 | KIAA0186 | KIAA0186 gene KIAA0186 gene product | NM_021067 | 9837 | 36232 | — |
| 39 | KIF14 | KIAA0042 gene KIAA0042 gene product | NM_014875 | 9928 | 3104 | — |
| | | KIAA0042 KIAA0042 gene product | | | | |
| 40 | KNSL6 | mitotic centromere-associated kinesin | NM_006845 | 11004 | 69360 | 604538 |
| | | mitotic centromere-associated kinesin d | | | | |
| | | kinesin-like 6 (mitotic centromere- | | | | |
| | | associated kinesin) HsMCAK mitotic | | | | |
| | | centromere-associated kinesin | | | | |
| 41 | KNSL7 | tu89b04.x1 kinesin-like protein 2 kinesin- | NM_020242 | 56992 | 150587 | — |
| | | like 7 | | | | |
| 42 | LGALS8 | HUMPCTA1A prostate carcinoma tumor | NM_006499 | 3964 | 4082 | 606099 |
| | | antigen (pcta-1) prostate carcinoma tumor | | | | |
| | | antigen (pcta-1) d lectin galactoside- | | | | |
| | | binding soluble 8 (galectin 8) prostate | | | | |
| | | carcinoma tumor antigen lectin, | | | | |
| | | galactoside-binding, soluble, 8 (galectin 8) | | | | |
| 43 | LISCH7 | DNA from chromosome 19-cosmid R30879 | NM_015925 | 51599 | 95697 | — |
| | | containing USF2 genomic sequence liver- | | | | |
| | | specific bHLH-Zip transcription factor | | | | |
| 44 | LMNB1 | lamin B1 gene lamin B1 lamin B1 | NM_005573 | 4001 | 89497 | 150340 |
| 45 | LMO4 | wb02d08.x1 LIM domain only 4 | NM_006769 | 8543 | 3844 | 603129 |
| 46 | MAN1A1 | zv92g08.r1 mannosidase alpha class 1A | NM_005907 | 4121 | 25253 | 604344 |
| | | member 1 EST | | | | |
| 47 | MCF2L | KIAA0362 gene MCF.2 cell line derived | NM_024979 | 23263 | 25515 | — |
| | | transforming sequence-like KIAA0362 | | | | |
| | | protein KIAA0362 | | | | |
| 48 | MCM4 | HSP1CDC21 P1-Cdc21 minichromosome | — | 4173 | 154443 | 602638 |
| | | maintenance deficient (S. cerevisiae) 4 | | | | |
| | | MCM4 minichromosome maintenance | | | | |
| | | deficient 4 (S. cerevisiae) | | | | |
| 49 | MKI67 | HSMKI67 mki67a (long type)antigen of | NM_002417 | 4288 | 80976 | 176741 |
| | | monoclonal antibody Ki-67 antigen | | | | |
| | | identified by monoclonal antibody Ki-67 | | | | |
| 50 | MLLT2 | AF-4 myeloidlymphoid or mixed-lineage | NM_005935 | 4299 | 114765 | 159557 |
| | | leukemia (trithorax (Drosophila) homolog) | | | | |
| | | translocated to 2 myeloid/lymphoid or | | | | |
| | | mixed-lineage leukemia (trithorax | | | | |
| | | (Drosophila) homolog); translocated to 2 | | | | |
| 51 | MPHOSPH6 | M-phase phosphoprotein mpp6 M-phase | NM_005792 | 10200 | 152720 | 605500 |
| | | phosphoprotein 6 M phase phosphoprotein; | | | | |
| | | MPP gene putative M-phase | | | | |
| | | phosphoprotein 6 | | | | |
| 52 | NF2 | NF2 = neurofibromatosis type 2 {alternatively | NM_000268 | 4771 | 902 | 607379 |
| | | spliced exon 15-16 form E3} colorectal | | | | |
| | | canc | | | | |
| 53 | NFIB | HSU70862 nuclear factor I B3 nuclear | NM_005596 | 4781 | 33287 | 600728 |
| | | factor I B3 d nuclear factor I/B | | | | |
| 54 | NMB | ws06b05.x1 neuromedin B | NM_021077 | 4828 | 83321 | 162340 |
| 55 | NPDC1 | tr90f10.x1 neural proliferation differentiation | NM_015392 | 56654 | 105547 | 605798 |
| | | and control 1 EST neural proliferation, | | | | |
| | | differentiation and control, 1 | | | | |
| 56 | NTF2 | genePP15 (placental protein 15) nuclear | NM_005796 | 10204 | 151734 | 605813 |
| | | transport factor 2 (placental protein 15) | | | | |
| | | placental protein 15 PP15 (AA 1-127) | | | | |
| | | nuclear transport factor 2 (placental protein | | | | |
| | | 15) | | | | |
| 57 | ORC6L | nz04b08.s1 origin recognition complex | NM_014321 | 23594 | 49760 | 607213 |
| | | subunit 6 (yeast homolog)-like EST origin | | | | |
| | | recognition complex, subunit 6 (yeast | | | | |
| | | homolog)-like | | | | |
| 58 | PCM1 | autoantigen pericentriol material 1 (PCM-1) | NM_006197 | 5108 | 75737 | 600299 |
| | | pericentriolar material 1 autoantigen; | | | | |
| | | pericentriol material 1 pericentriolar | | | | |
| | | material 1 | | | | |
| 59 | PNAS-4 | wq94g10.x1 apoptosis-related protein | NM_016076 | 51029 | 42409 | — |
| | | PNAS-4 (PNAS-4) d CGI-146 protein EST | | | | |
| 60 | PPP3CC | wh92e05.x1 protein phosphatase 3 | NM_005605 | 5533 | 75206 | 114107 |
| | | (formerly 2B) catalytic subunit gamma | | | | |
| | | isoform (calcineurin A gamma) EST protein | | | | |
| | | phosphatase 3 (formerly 2B), catalytic | | | | |
| | | subunit, | | | | |
| 61 | PPT2 | inactive palmitoyl-protein thioesterase-2i | NM_005155 | 9374 | 81737 | 603298 |
| | | (PPT2) inactive palmitoyl-protein | | | | |
| | | thioesterase-2i (PPT2) d palmitoyl-protein | | | | |
| | | thioesterase 2 | | | | |
| 62 | PRKX | PRKY protein protein kinase X-linked | NM_005044 | 5613 | 56336 | 300083 |
| | | protein kinase Y-linked protein kinase, X- | | | | |
| | | linked | | | | |
| 63 | PTTG1 | zx55e01.r1 pituitary tumor-transforming | NM_004219 | 9232 | 252587 | 604147 |
| | | protein 1 pituitary tumor-transforming 1 | | | | |
| 64 | RAB31 | zp82b12.s1 RAB31 member RAS | NM_006868 | 11031 | 223025 | 605694 |
| | | oncogene family RAB31, member RAS | | | | |
| | | oncogene family | | | | |
| 65 | RAB6KIFL | wi18c04.x1 RAB6 interacting kinesin-like | NM_005733 | 10112 | 73625 | 605664 |
| | | (rabkinesin6) EST RAB6 interacting, | | | | |
| | | kinesin-like (rabkinesin6) | | | | |
| 66 | RBSK | wj29b03.x1 ESTs Weakly similar to/ | NM_022128 | 64080 | 11916 | — |
| | | prediction EST ribokinase | | | | |
| 67 | RCP | ni63c11.s1 hypothetical protein FLJ22622 | NM_025151 | 80223 | 324841 | — |
| | | Homo sapiens cDNA: FLJ22622 fis clone | | | | |
| | | HSI05669 EST | | | | |
| 68 | RFC4 | HUMACT1A replication factor C 37-kDa | NM_002916 | 5984 | 35120 | 102577 |
| | | subunit replication factor C (activator 1) 4 | | | | |
| | | (37 kD) RFC; Activator 1 replicative | | | | |
| | | polymerase accessory protein; activator 1 | | | | |
| | | replication factor C, 37-kDa subunit | | | | |
| 69 | RGS5 | regulator of G-protein signalling 5 (RGS5) d | NM_003617 | 8490 | 24950 | 603276 |
| 70 | RRAS2 | Ras-Like Protein Tc21 oncogene TC21 | NM_012250 | 22800 | 206097 | 600098 |
| | | related RAS viral (r-ras) oncogene homolog 2 | | | | |
| 71 | SCRG1 | scrapie responsive protein 1 scrapie | NM_007281 | 11341 | 7122 | 603163 |
| | | responsive protein 1 scrapie responsive | | | | |
| | | protein 1; ScRG-1 gene scrapie responsive | | | | |
| | | protein 1 | | | | |
| 72 | SIAT7E | zf79e09.s1 similar to sialyltransferase 7 | NM_030965 | 81849 | 26981 | — |
| | | ((alpha-N-acetylneuraminyl 2 3- | | | | |
| | | betagalactosyl-1 3)-N-acetyl | | | | |
| | | galactosaminide alpha-2 6- | | | | |
| | | sialyltransferase) E ESTs | | | | |
| 73 | SLC2A10 | af28a03.s1 solute carrier family 2 | NM_030777 | 81031 | 17863 | 606145 |
| | | (facilitated glucose transporter) member 10 | | | | |
| | | ESTs | | | | |
| 74 | SLC6A8 | GABA noradrenaline transporter solute | NM_005629 | 6535 | 187958 | 300036 |
| | | carrier family 6 (neurotransmitter | | | | |
| | | transporter creatine) member 8 | | | | |
| | | GABA/noradrenaline transporter solute | | | | |
| | | carrier family 6 (neurotransmitter | | | | |
| | | transporter, | | | | |
| 75 | SPTBN2 | beta III spectrin (SPTBN2) spectrin beta | NM_006946 | 6712 | 26915 | 604985 |
| | | non-erythrocytic 2 membrane skeletal | | | | |
| | | protein; beta spectrin nonerythroid form 2 | | | | |
| | | beta III spectrin spectrin, beta, non- | | | | |
| | | erythrocytic 2 | | | | |
| 76 | TFAP2C | transcription factor ERF-1 transcription | NM_003222 | 7022 | 61796 | 601602 |
| | | factor AP-2 gamma (activating enhancer- | | | | |
| | | binding protein 2 gamma) | | | | |
| 77 | TGFA | transforming growth factor alpha | NM_003236 | 7039 | 170009 | 190170 |
| | | transforming growth factor, alpha | | | | |
| 78 | TM4SF1 | tj34g07.x1 transmembrane 4 superfamily | NM_014220 | 4071 | 3337 | 191155 |
| | | member 1 | | | | |
| 79 | TSLRP | testis specific leucine rich repeat protein | NM_012472 | 23639 | 57693 | — |
| | | (TSLRP) testis specific leucine rich repeat | | | | |
| | | protein testis specific leucine rich repeat | | | | |
| | | protein | | | | |
| 80 | TST | rhodanese thiosulfate sulfurtransferase | NM_003312 | 7263 | 248267 | 180370 |
| | | (rhodanese) rhodanese thiosulfate:cyanide | | | | |
| | | sulfurtransferase thiosulfate | | | | |
| | | sulfurtransferase (rhodanese) | | | | |
| 81 | UCHL1 | protein gene product (PGP) 9.5 ubiquitin | NM_004181 | 7345 | 76118 | 191342 |
| | | carboxyl-terminal esterase L1 (ubiquitin | | | | |
| | | thiolesterase) neuroendocrine marker | | | | |
| | | protein PGP 9.5 (AA 1-212) ubiquitin | | | | |
| | | carboxyl-terminal esterase L1 (ubiquitin | | | | |
| | | thiolesterase) | | | | |
| 82 | WAC | at11d08.x1 lectin galactoside-binding | NM_018072 | 55127 | 4082 | — |
| | | soluble 8 (galectin 8) hypothetical protein | | | | |
| | | PRO1741 EST lectin, galactoside-binding, | | | | |
| | | soluble, 8 (galectin 8) | | | | |
| 83 | XIAP | KIAA0590 protein KIAA0590 gene product | NM_014714 | 9742 | 111862 | — |
| | | KIAA0590 protein KIAA0590 gene product | | | | |
| 84 | ZAP128 | (clone zap128) of cds peroxisomal long- | NM_006821 | 10965 | 299629 | — |
| | | chain acyl-coA thioesterase peroxisomal | | | | |
| | | long-chain acyl-coA thioesterase; putative | | | | |
| | | protein ORF; putative | | | | |

### EXAMPLE 2

### Expression Profiling Utilizing DNA Microarrays

Expression profiling can bee carried out using the Affymetrix Array Technology. By hybridization of mRNA to such a DNA-array or DNA-Chip, it is possible to identify the expression value of each transcripts due to signal intensity at certain position of the array. Usually these DNA-arrays are produced by spotting of cDNA, oligonucleotides or subcloned DNA fragments. In case of Affymetrix technology app. 400.000 individual oligonucleotide sequences were synthesized on the surface of a silicon wafer at distinct positions. The minimal length of oligomers is 12 nucleotides, preferable 25 nucleotides or full length of the questioned transcript. Expression profiling may also be carried out by hybridization to nylon or nitro-cellulose membrane bound DNA or oligonucleotides. Detection of signals derived from hybridization may be obtained by either colorimetric, fluorescent, electrochemical, electronic, optic or by radioactive readout. Detailed description of array construction have been mentioned above and in other patents cited. To determine the quantitative and qualitative changes in the gene expression of certain breast cancer specimens, RNA from tumor tissue extracted prior to any chemotherapy has to be compared among each other individually and/or to RNA extracted from benign tissue (e.g. epithelial breast tissue, or micro dissected ductal tissue) on the basis of expression profiles for the whole transcriptome. With minor modifications, the sample preparation protocol followed the Affymetrix GeneChip Expression Analysis Manual (Santa Clara, Calif.). Total RNA extraction and isolation from tumor or benign tissues, biopsies, cell isolates or cell containing body fluids can be performed by using TRIzol (Life Technologies, Rockville, Md.) and Oligotex mRNA Midi kit (Qiagen, Hilden, Germany), and an ethanol precipitation step should be carried out to bring the concentration to 1 mg/ml. Using 5-10 mg of mRNA to create double stranded cDNA by the SuperScript system (Life Technologies). First strand cDNA synthesis was primed with a T7-(dT24) oligonucleotide. The cDNA can be extracted with phenol/chloroform and precipitated with ethanol to a final concentration of 1 mg/ml. From the generated cDNA, cRNA can be synthesized using Enzo's (Enzo Diagnostics Inc., Farmingdale, N.Y.) in vitro Transcription Kit. Within the same step the cRNA can be labeled with biotin nucleotides Bio-11-CTP and Bio-16-UTP (Enzo Diagnostics Inc., Farmingdale, N.Y.). After labeling and cleanup (Qiagen, Hilden (Germany) the cRNA then should be fragmented in an appropriated fragmentation buffer (e.g., 40 mM Tris-Acetate, pH 8.1, 100 mM KOAc, 30 mM MgOAc, for 35 minutes at 94° C.). As per the Affymetrix protocol, fragmented cRNA should be hybridized on the HG_U133 arrays (as used herein), comprising app. 40.000 probed transcripts each, for 24 hours at 60 rpm in a 45° C. hybridization oven. After Hybridization step the chip surfaces have to be washed and stained with streptavidin phycoerythrin (SAPE; Molecular Probes, Eugene, Oreg.) in Affymetrix fluidics stations. To amplify staining, a second labeling step can be introduced, which is recommended but not compulsive. Here one should add SAPE solution twice with an antistreptavidin biotinylated antibody. Hybridization to the probe arrays may be detected by fluorometric scanning (Hewlett Packard Gene Array Scanner; Hewlett Packard Corporation, Palo Alto, Calif.).

After hybridization and scanning, the microarray images can be analyzed for quality control, looking for major chip defects or abnormalities in hybridization signal. Therefor either Affymetrix GeneChip MAS 5.0 Software or other microarray image analysis software can be utilized. Primary data analysis should be carried out by software provided by the manufacturer. In case of the genes analyses in one embodiment of this invention the primary data have been analyzed by further bioinformatic tools and additional filter criteria as described in example 3.

### EXAMPLE 3

### Data Analysis from Expression Profiling Experiments

According to Affymetrix measurement technique (Affymetrix GeneChip Expression Analysis Manual, Santa Clara, Calif.) a single gene expression measurement on one chip yields the average difference value and the absolute call. Each chip contains 16-20 oligonucleotide probe pairs per gene or cDNA clone. These probe pairs include perfectly matched sets and mismatched sets, both of which are necessary for the calculation of the average difference, or expression value, a measure of the intensity difference for each probe pair, calculated by subtracting the intensity of the mismatch from the intensity of the perfect match. This takes into consideration variability in hybridization among probe pairs and other hybridization artifacts that could affect the fluorescence intensities. The average difference is a numeric value supposed to represent the expression value of that gene. The absolute call can take the values ‘A’ (absent), ‘M’ (marginal), or ‘P’ (present) and denotes the quality of a single hybridization. We used both the quantitative information given by the average difference and the qualitative information given by the absolute call to identify the genes which are differentially expressed in biological samples from individuals with breast cancer versus biological samples from the normal population. With other algorithms than the Affymetrix one we have obtained different numerical values representing the same expression values and expression differences upon comparison.

The differential expression E in one of the breast cancer groups compared to the normal population is calculated as follows. Given n average difference values d1, d2, . . . , dn in the breast cancer population and m average difference values c1, c2, . . . , cm in the population of normal individuals, it is computed by the equation:

$$E ≡ exp ( \frac{1}{m} {\sum }_{i = 1}^{m} ln ( {c}_{i} ) - \frac{1}{n} {\sum }_{i = 1}^{n} ln ( {d}_{i} ) ) \qquad ( equation 1 )$$

If dj<50 or ci<50 for one or more values of i and j, these particular values ci and/or dj are set to an “artificial” expression value of 50. These particular computation of E allows for a correct comparison to TaqMan results.

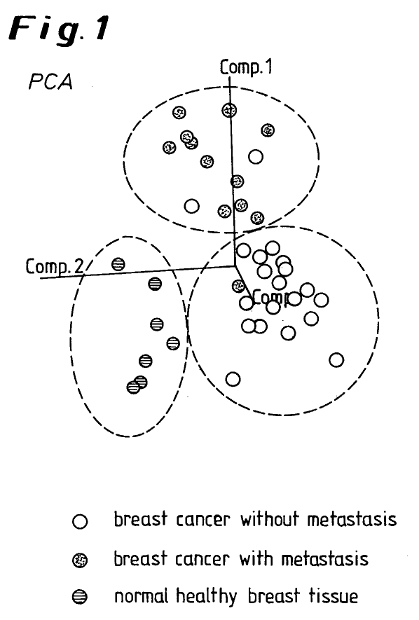

A gene is called up-regulated in breast cancer in tissues responding or non-responding to chemotherapy, if E>=average change factor given in Table 2 and if the number of absolute calls equal to ‘P’ in the breast cancer population is greater than n/2. The average fold change factors in Table 2 are given for those tumor population developing distant metastasis despite of a given chemotherapy (sample group 1), those do not develop distant metastasis with in the first 50 month post therapy (sample group 3) or those tissues without any pathological signs of a tumor (sample group 1). Fold changes greater than 1 refers to an increase in gene expression in the first named tissue sample compared to the second. This regulation factors are mean values and may differ individually, here the combined profiles of all 84 genes listed in Table 1 in a cluster analysis or a principle component analysis (PCA) will indicate the classification group for such sample (See FIG. 1 for representative PCA with 84 genes and three classes). By a PCA one will identify the major components (Eigengenes or Eigenvectors) which do discriminate the samples analyzed.

According to the above, a gene is called down-regulated in one tumor class versus another or normal breast tissue if E<=minimal change factor given in Table 2 and if the number of absolute calls equal to ‘P’ in the breast cancer population is greater than n/2. Values smaller than 1 describe an decreased expression of the given gene.

The average fold change factors given in Table 2 indicate also the relative up- and down-regulation of those gene indicative of tumor presence. The final list of differentially regulated genes consists of all up-regulated and all down-regulated genes in biological samples from individuals with breast cancer versus biological samples from the normal population or of an individual response pattern. Those genes on this list which are interesting for a diagnostic or pharmaceutical application were finally validated by quantitative real time RT-PCR (see Example 1). If a good correlation between the expression values/behavior of a transcript could be observed with both techniques, such a gene is listed in Table 1.

### Data Filtering:

Raw data were acquired using Microsuite 5.0 software of Affymetrix and normalized following a standard practice of scaling the average of all gene signal intensities to a common arbitrary value. 59 Genes corresponding to Affymetrix controls (housekeeping genes, etc.) were removed from the analysis. The only exception has been done for the genes for GAPDH and Beta-actin, which expression levels were used for the normalization purposes. One hundred genes, which expression levels are routinely used in order to normalized between HG-U133A and HG-U133B GeneChips, were also removed from the analysis. Genes with potentially high levels of noise (81 probe sets), which is observed for genes with low absolute expression values (genes, which expression levels did not achieve 30 RLU (TGT=100) through all experiments), were removed from the data set. The remaining genes were preprocessed to eliminate the genes (3196 probe sets) whose signal intensities were not significantly different from their background levels and thus labeled as “Absent” by Affymetrix MicroSuite 5.0 in all experiments. We eliminated genes that were not present in at least 10% of samples (3841 probe sets). Data for remaining 15,006 probe sets were subsequently analysed by statistical methods.

### Statistical Analysis:

In order to optimize prediction of non responding tumor samples one may use this class from the training cohort and run multiple statistical tests, suitable for group comparison including nonparametric Wilcoxon rank sum test, two-sample independent Students' t-test, Welch test, Kolmogorov-Smirnov test (for variance), and SUM-Rank test (see Table 3). As listed in Table 2 one can identify such genes with a differential expression in the metastasis group vs. The non metastasis group and a significance level (p-value) below 0.05. Hereby we identified 84 significantly differentially regulated genes displayed in Table 1.

Additionally one may apply correction for multiple testing errors such as Benjamini-Hochberg and may apply tests for False Discovery Detection such as permutations with Bootstrap or Jack-knife algorithms.

TABLE 2
Relative expression of 84 genes in breastcancers developing distant metestasis as
compared to breast cancers not developing distant metastasis within 50 month.
(and as compared to normal healthy tissue)

| | | | SAMPLE | SAMPLE | | | | |
|---|---|---|---|---|---|---|---|---|
| | | | GROUP 2 | GROUP 3 | REL. | REL. | REL. | |
| | | SAMPLE | breast | breast | FC | FC | FC | |
| | | GROUP 1 | cancer | cancer | group | group 3 | group 3 | |
| Gene | Ref. | normal | with | without | 2 vs. | vs. | vs. | |
| Symbol | Sequences | breast | metastasis | metastasis | group 1 | group 1 | group 2 | regulation |
| MAN1A1 | NM_005907 | 308.69 | 106.73 | 259.57 | 0.35 | 0.84 | 2.43 | up |
| CEACAM6 | NM_002483 | 32.38 | 231.02 | 521.27 | 7.13 | 16.10 | 2.26 | up |
| SLC2A10 | NM_030777 | 127.96 | 154.34 | 335.44 | 1.21 | 2.62 | 2.17 | up |
| MCF2L | NM_024979 | 92.55 | 51.92 | 103.41 | 0.56 | 1.12 | 1.99 | up |
| PPT2 | NM_005155 | 65.35 | 20.64 | 40.49 | 0.32 | 0.62 | 1.96 | up |
| TSLRP | NM_012472 | 39.06 | 30.01 | 58.76 | 0.77 | 1.50 | 1.96 | up |
| RGS5 | NM_003617 | 542.22 | 174.06 | 331.58 | 0.32 | 0.61 | 1.91 | up |
| HEMK | NM_016173 | 32.44 | 25.87 | 48.96 | 0.80 | 1.51 | 1.89 | up |
| KIAA0590 | NM_014714 | 29.88 | 19.99 | 37.80 | 0.67 | 1.26 | 1.89 | up |
| RCP | NM_025151 | 85.13 | 144.72 | 273.29 | 1.70 | 3.21 | 1.89 | up |
| RAB31 | NM_006868 | 164.10 | 352.14 | 652.04 | 2.15 | 3.97 | 1.85 | up |
| GLUL | NM_002065 | 384.26 | 397.29 | 716.53 | 1.03 | 1.86 | 1.80 | up |
| ZAP128 | NM_006821 | 215.96 | 98.96 | 177.82 | 0.46 | 0.82 | 1.80 | up |
| GALNAC4S- | NM_014863 | 159.32 | 167.06 | 297.25 | 1.05 | 1.87 | 1.78 | up |
| 6ST | | | | | | | | |
| TST | NM_003312 | 162.80 | 91.38 | 160.38 | 0.56 | 0.99 | 1.76 | up |
| GATM | NM_001482 | 133.77 | 72.05 | 125.87 | 0.54 | 0.94 | 1.75 | up |
| PCM1 | NM_006197 | 216.38 | 132.13 | 225.68 | 0.61 | 1.04 | 1.71 | up |
| RBSK | NM_022128 | 34.00 | 28.06 | 46.58 | 0.83 | 1.37 | 1.66 | up |
| MLLT2 | NM_005935 | 527.65 | 260.35 | 399.54 | 0.49 | 0.76 | 1.53 | up |
| FLJ12949 | NM_023008 | 24.28 | 24.65 | 37.60 | 1.02 | 1.55 | 1.52 | up |
| PPP3CC | NM_005605 | 46.87 | 22.34 | 33.45 | 0.48 | 0.71 | 1.50 | up |
| NPDC1 | NM_015392 | 173.96 | 159.40 | 235.61 | 0.92 | 1.35 | 1.48 | up |
| LGALS8 | NM_006499 | 54.92 | 34.48 | 50.68 | 0.63 | 0.92 | 1.47 | up |
| FLJ22029 | NM_024949 | 22.74 | 24.01 | 35.14 | 1.06 | 1.55 | 1.46 | up |
| WAC | NM_018072 | 191.91 | 147.98 | 214.49 | 0.77 | 1.12 | 1.45 | up |
| ORC6L | NM_014321 | 49.36 | 53.85 | 37.23 | 1.09 | 0.75 | 0.69 | down |
| FLJ21313 | NM_023927 | 215.79 | 113.12 | 78.07 | 0.52 | 0.36 | 0.69 | down |
| NTF2 | NM_005796 | 208.79 | 237.26 | 163.18 | 1.14 | 0.78 | 0.69 | down |
| CDT1 | NM_030928 | 19.42 | 43.16 | 29.34 | 2.22 | 1.51 | 0.68 | down |
| BTBD3 | NM_014962 | 154.16 | 140.69 | 95.43 | 0.91 | 0.62 | 0.68 | down |
| KARS | NM_005548 | 698.75 | 944.54 | 639.45 | 1.35 | 0.92 | 0.68 | down |
| CDC25B | NM_004358 | 166.61 | 248.71 | 167.47 | 1.49 | 1.01 | 0.67 | down |
| MKI67 | NM_002417 | 44.02 | 88.96 | 59.54 | 2.02 | 1.35 | 0.67 | down |
| MPHOSPH6 | NM_005792 | 180.73 | 236.52 | 158.17 | 1.31 | 0.88 | 0.67 | down |
| NF2 | NM_000268 | 31.87 | 33.02 | 22.04 | 1.04 | 0.69 | 0.67 | down |
| FLJ20364 | NM_017785 | 33.58 | 46.30 | 30.70 | 1.38 | 0.91 | 0.66 | down |
| MCM4 | — | 34.89 | 117.30 | 77.77 | 3.36 | 2.23 | 0.66 | down |
| LOC51029 | NM_016076 | 42.78 | 140.37 | 92.65 | 3.28 | 2.17 | 0.66 | down |
| LMO4 | NM_006769 | 25.48 | 39.43 | 25.34 | 1.55 | 0.99 | 0.64 | down |
| ENO1 | NM_001428 | 351.52 | 859.22 | 547.65 | 2.44 | 1.56 | 0.64 | down |
| KNSL7 | NM_020242 | 14.96 | 39.17 | 24.90 | 2.62 | 1.66 | 0.64 | down |
| DDA3 | NM_001826 | 56.79 | 90.47 | 57.46 | 1.59 | 1.01 | 0.64 | down |
| RRAS2 | NM_012250 | 51.89 | 45.85 | 28.84 | 0.88 | 0.56 | 0.63 | down |
| ADAM17 | NM_003183 | 32.32 | 35.70 | 22.41 | 1.10 | 0.69 | 0.63 | down |
| TFAP2C | NM_003222 | 52.01 | 45.13 | 28.23 | 0.87 | 0.54 | 0.63 | down |
| LOC64116 | NM_022154 | 79.94 | 91.43 | 57.16 | 1.14 | 0.72 | 0.63 | down |
| KIAA0042 | NM_014875 | 20.11 | 63.54 | 39.71 | 3.16 | 1.97 | 0.62 | down |
| UCHL1 | NM_004181 | 177.01 | 153.65 | 95.60 | 0.87 | 0.54 | 0.62 | down |
| GPR56 | NM_005682 | 248.33 | 250.48 | 155.58 | 1.01 | 0.63 | 0.62 | down |
| LMNB1 | NM_005573 | 26.05 | 83.17 | 51.43 | 3.19 | 1.97 | 0.62 | down |
| MGC3184 | NM_030965 | 37.90 | 67.62 | 41.51 | 1.78 | 1.10 | 0.61 | down |
| KNSL6 | NM_006845 | 62.18 | 157.38 | 95.67 | 2.53 | 1.54 | 0.61 | down |
| ASK | NM_006716 | 25.59 | 59.10 | 35.92 | 2.31 | 1.40 | 0.61 | down |
| RAB6KIFL | NM_005733 | 25.20 | 121.59 | 73.87 | 4.83 | 2.93 | 0.61 | down |
| FLJ10079 | NM_017990 | 29.83 | 40.14 | 24.27 | 1.35 | 0.81 | 0.60 | down |
| HMMR | NM_012484 | 21.86 | 67.57 | 40.63 | 3.09 | 1.86 | 0.60 | down |
| NMB | NM_021077 | 172.95 | 96.09 | 57.25 | 0.56 | 0.33 | 0.60 | down |
| BTG3 | NM_006806 | 147.50 | 193.33 | 113.37 | 1.31 | 0.77 | 0.59 | down |
| LISCH7 | NM_015925 | 115.79 | 270.47 | 156.62 | 2.34 | 1.35 | 0.58 | down |
| PTTG1 | NM_004219 | 132.24 | 353.40 | 198.23 | 2.67 | 1.50 | 0.56 | down |
| FLJ10156 | NM_019013 | 33.28 | 78.56 | 43.77 | 2.36 | 1.32 | 0.56 | down |
| NFIB | NM_005596 | 126.29 | 95.06 | 52.92 | 0.75 | 0.42 | 0.56 | down |
| APRT | NM_000485 | 164.36 | 304.70 | 168.65 | 1.85 | 1.03 | 0.55 | down |
| TGFA | NM_003236 | 62.34 | 43.69 | 24.03 | 0.70 | 0.39 | 0.55 | down |
| PRKX | NM_005044 | 44.14 | 78.73 | 42.36 | 1.78 | 0.96 | 0.54 | down |
| CENPF | NM_016343 | 39.59 | 205.96 | 110.34 | 5.20 | 2.79 | 0.54 | down |
| RFC4 | NM_002916 | 93.11 | 264.11 | 139.05 | 2.84 | 1.49 | 0.53 | down |
| FLJ20354 | NM_017779 | 20.88 | 47.77 | 24.81 | 2.29 | 1.19 | 0.52 | down |
| ANKT | NM_016359 | 9.14 | 90.27 | 46.77 | 9.88 | 5.12 | 0.52 | down |
| GTSE1 | NM_016426 | 22.70 | 55.93 | 28.83 | 2.46 | 1.27 | 0.52 | down |
| CCNB1 | NM_031966 | 43.59 | 168.62 | 85.21 | 3.87 | 1.95 | 0.51 | down |
| CXADR | NM_001338 | 196.41 | 383.00 | 188.80 | 1.95 | 0.96 | 0.49 | down |
| KCNK5 | NM_003740 | 33.90 | 51.36 | 24.66 | 1.51 | 0.73 | 0.48 | down |
| SPTBN2 | NM_006946 | 18.01 | 46.34 | 21.71 | 2.57 | 1.21 | 0.47 | down |
| SLC6A8 | NM_005629 | 63.61 | 298.19 | 133.16 | 4.69 | 2.09 | 0.45 | down |
| CDKN2A | NM_000077 | 35.93 | 97.01 | 42.99 | 2.70 | 1.20 | 0.44 | down |
| DUSP9 | NM_001395 | 21.38 | 47.49 | 20.35 | 2.22 | 0.95 | 0.43 | down |
| TM4SF1 | NM_014220 | 443.13 | 441.10 | 184.84 | 1.00 | 0.42 | 0.42 | down |
| E2-EPF | NM_014501 | 113.76 | 612.43 | 254.45 | 5.38 | 2.24 | 0.42 | down |
| SCRG1 | NM_007281 | 66.64 | 74.50 | 30.52 | 1.12 | 0.46 | 0.41 | down |
| KIAA0186 | NM_021067 | 33.73 | 149.70 | 60.72 | 4.44 | 1.80 | 0.41 | down |
| FOLR1 | NM_000802 | 131.90 | 104.33 | 40.48 | 0.79 | 0.31 | 0.39 | down |
| CTSL2 | NM_001333 | 67.88 | 117.17 | 36.74 | 1.73 | 0.54 | 0.31 | down |
| DSG3 | NM_001944 | 73.61 | 108.22 | 33.80 | 1.47 | 0.46 | 0.31 | down |

TABLE 3
p-values for statistical significance for 84 genes predicting therapeutic success.

| GENE_SYMBOL | REF. SEQUENCES | T-TEST | WELCH | WILCOXON |
|---|---|---|---|---|
| SLC2A10 | NM_030777 | 1.79E−05 | 9.25E−04 | 4.54E−04 |
| FLJ20354 | NM_017779 | 2.20E−05 | 1.90E−04 | 1.22E−04 |
| DDA3 | NM_001826 | 2.49E−05 | 2.81E−04 | 2.38E−04 |
| E2-EPF | NM_014501 | 3.84E−05 | 2.59E−04 | 2.38E−04 |
| KIAA0186 | NM_021067 | 6.69E−05 | 5.54E−04 | 4.54E−04 |
| FLJ10156 | NM_019013 | 8.82E−05 | 0.001112 | 5.82E−04 |
| ZAP128 | NM_006821 | 1.28E−04 | 4.54E−04 | 3.09E−04 |
| CTSL2 | NM_001333 | 1.42E−04 | 0.004204 | 0.001202 |
| APRT | NM_000485 | 1.78E−04 | 0.001993 | 4.54E−04 |
| CCNB1 | NM_031966 | 1.79E−04 | 9.98E−04 | 0.001352 |
| RFC4 | NM_002916 | 2.91E−04 | 0.003498 | 0.00267 |
| FLJ20364 | NM_017785 | 4.03E−04 | 3.19E−05 | 0.001202 |
| PTTG1 | NM_004219 | 4.10E−04 | 0.002346 | 0.001068 |
| MLLT2 | NM_005935 | 4.44E−04 | 0.001047 | 0.001202 |
| PPT2 | NM_005155 | 4.68E−04 | 0.00595 | 0.006239 |
| GALNAC4S-6ST | NM_014863 | 4.83E−04 | 8.35E−04 | 0.001068 |
| MAN1A1 | NM_005907 | 5.00E−04 | 1.04E−04 | 5.82E−04 |
| BTBD3 | NM_014962 | 5.59E−04 | 0.001462 | 0.002979 |
| KIAA0590 | NM_014714 | 6.00E−04 | 6.09E−04 | 4.54E−04 |
| NMB | NM_021077 | 6.33E−04 | 2.32E−04 | 5.82E−04 |
| ANKT | NM_016359 | 7.79E−04 | 7.99E−04 | 5.14E−04 |
| BTG3 | NM_006806 | 8.68E−04 | 9.77E−04 | 0.001352 |
| DUSP9 | NM_001395 | 0.001043 | 0.001354 | 4.54E−04 |
| HEMK | NM_016173 | 0.001067 | 0.008184 | 0.005631 |
| KNSL7 | NM_020242 | 0.001098 | 5.36E−04 | 8.41E−04 |
| CDKN2A | NM_000077 | 0.001331 | 0.009036 | 0.01025 |
| SCRG1 | NM_007281 | 0.001333 | 0.01169 | 4.54E−04 |
| KARS | NM_005548 | 0.001438 | 0.009492 | 0.005077 |
| CDT1 | NM_030928 | 0.0018 | 0.001508 | 0.001703 |
| LMNB1 | NM_005573 | 0.001846 | 4.96E−04 | 0.001909 |
| FLJ10079 | NM_017990 | 0.0019 | 0.01041 | 0.002137 |
| CENPF | NM_016343 | 0.001908 | 0.01038 | 0.01025 |
| PRKX | NM_005044 | 0.001965 | 0.005998 | 0.003321 |
| RBSK | NM_022128 | 0.002226 | 0.003793 | 0.004573 |
| SLC6A8 | NM_005629 | 0.002413 | 0.006489 | 0.004573 |
| MCF2L | NM_024979 | 0.002434 | 0.009569 | 0.008431 |
| LMO4 | NM_006769 | 0.002499 | 9.47E−04 | 0.003699 |
| DSG3 | NM_001944 | 0.002509 | 0.003476 | 0.006239 |
| TGFA | NM_003236 | 0.003116 | 0.007069 | 0.002979 |
| TM4SF1 | NM_014220 | 0.003336 | 0.007194 | 0.01025 |
| KNSL6 | NM_006845 | 0.003369 | 0.008465 | 0.009303 |
| RRAS2 | NM_012250 | 0.003427 | 0.001855 | 0.004115 |
| ENO1 | NM_001428 | 0.0036 | 0.009001 | 0.005631 |
| MKI67 | NM_002417 | 0.003717 | 0.01721 | 0.01498 |
| MGC3184 | NM_030965 | 0.004221 | 0.002031 | 0.001352 |
| NTF2 | NM_005796 | 0.004554 | 0.00127 | 0.002979 |
| TSLRP | NM_012472 | 0.004642 | 0.008277 | 0.004115 |
| HMMR | NM_012484 | 0.005021 | 0.01257 | 0.002979 |
| RGS5 | NM_003617 | 0.005034 | 0.002572 | 0.001703 |
| PCM1 | NM_006197 | 0.005075 | 0.006982 | 0.01129 |
| CXADR | NM_001338 | 0.005338 | 0.001932 | 0.001518 |
| FOLR1 | NM_000802 | 0.005372 | 0.02658 | 0.004573 |
| LOC64116 | NM_022154 | 0.005451 | 0.0136 | 0.007634 |
| NPDC1 | NM_015392 | 0.006615 | 0.03824 | 0.0331 |
| KCNK5 | NM_003740 | 0.007629 | 0.02425 | 0.0235 |
| ADAM17 | NM_003183 | 0.00829 | 0.001635 | 0.006905 |
| CDC25B | NM_004358 | 0.008483 | 0.0177 | 0.01642 |
| SPTBN2 | NM_006946 | 0.008691 | 0.003847 | 0.01129 |
| GLUL | NM_002065 | 0.008768 | 0.009478 | 0.01642 |
| KIAA0042 | NM_014875 | 0.009176 | 0.01 | 0.01364 |
| WAC | NM_018072 | 0.009256 | 0.02867 | 0.01969 |
| RAB6KIFL | NM_005733 | 0.00993 | 0.02459 | 0.006905 |
| PPP3CC | NM_005605 | 0.01025 | 0.01181 | 0.01498 |
| ORC6L | NM_014321 | 0.01121 | 0.008294 | 0.01025 |
| FLJ21313 | NM_023927 | 0.01153 | 0.01009 | 0.009303 |
| UCHL1 | NM_004181 | 0.01215 | 0.01744 | 0.004573 |
| GPR56 | NM_005682 | 0.01243 | 0.02093 | 0.01642 |
| RAB31 | NM_006868 | 0.0125 | 0.006138 | 0.01242 |
| MPHOSPH6 | NM_005792 | 0.01329 | 0.007349 | 0.01129 |
| FLJ22029 | NM_024949 | 0.01345 | 0.03506 | 0.02795 |
| LGALS8 | NM_006499 | 0.01406 | 0.02723 | 0.02152 |
| GATM | NM_001482 | 0.01459 | 0.01213 | 0.01799 |
| NFIB | NM_005596 | 0.01807 | 0.01125 | 0.01364 |
| LISCH7 | NM_015925 | 0.01842 | 0.02284 | 0.01129 |
| LOC51029 | NM_016076 | 0.01921 | 0.03229 | 0.02564 |
| FLJ12949 | NM_023008 | 0.01925 | 0.03729 | 0.02564 |
| GTSE1 | NM_016426 | 0.01954 | 0.04557 | 0.01642 |
| NF2 | NM_000268 | 0.02133 | 0.004181 | 0.01642 |
| ASK | NM_006716 | 0.02286 | 0.04786 | 0.0235 |
| CEACAM6 | NM_002483 | 0.02334 | 0.03772 | 0.04963 |
| RCP | NM_025151 | 0.0306 | 0.02368 | 0.0235 |
| MCM4 | — | 0.03473 | 0.03093 | 0.03043 |
| TFAP2C | NM_003222 | 0.03473 | 0.0413 | 0.03904 |
| TST | NM_003312 | 0.04396 | 0.01513 | 8.41E−04 |

### EXAMPLE 4

### Statistical Relevance of 84 Genes Differentially Expressed in Breast Cancers Developing Distant Metestasis as Compared to Breast Cancers not Developing Distant Metastasis within 50 Month.Prediction of Tumor Classes Based on Expression Profiles

While as those algorithms described in Example 3 can be implemented in a certain kernel to classify samples according to their specific gene expression into two classes another approach can be taken to predict class membership by implementation of a k-NN classification. The method of k-Nearest Neighbors (k-NN), proposed by T. M. Cover and P. E. Hart, an important approach to nonparametric classification, is quite easy and efficient. Partly because of its perfect mathematical theory, NN method develops into several variations. As we know, if we have infinitely many sample points, then the density estimates converge to the actual density function. The classifier becomes the Bayesian classifier if the large-scale sample is provided. But in practice, given a small sample, the Bayesian classifier usually fails in the estimation of the Bayes error especially in a high-dimensional space, which is called the disaster of dimension. Therefore, the method of k-NN has a great pity that the sample space must be large enough.

In k-nearest-neighbor classification, the training data set is used to classify each member of a “target” data set. The structure of the data is that there is a classification (categorical) variable of interest (e.g. “metastasizing breast tumors” (sample group 2) or “non-metastazising breast tumors” (sample group 3)), and a number of additional predictor variables (gene expression values). Generally speaking, the algorithm is as follows:

1. For each sample in the data set to be classified, locate the k nearest neighbors of the training data set. A Euclidean distance measure or a correlation analysis can be used to calculate how close each member of the training set is to the target sample that is being examined.
2. Examine the k nearest neighbors—which classification do most of them belong to?
3. Assign this category to the sample being examined.
4. Repeat this procedure steps 1 to 3 for the remaining samples in the target set.

Of course the computing time goes up as k goes up, but the advantage is that higher values of k provide smoothing that reduces vulnerability to noise in the training data. In practical applications, typically, k is in units or tens rather than in hundreds or thousands. In this disclosure we have used a k=3.

The “nearest neighbors” are determined if given the considered the vector and the distance measurement. Given a training set of expression values for a certain number of samples

T={(x1,y1), (x2,y2), . . . , (xm,ym)}, to determine the class of the input vector x.

The most special case is the k-NN method, while k=1, which just searches the one nearest neighbor:

j=argmin//x−xi//

then, (x, yj) is the solution.

For estimation on the error rate of this classification the following considerations could be made:

A training set T={(x1, y1), (x2, y2), . . . , (xm, ym)} is called (k, d %)-stable if the error rate of k-NN method is d %, where d % is the empirical error rate from independent experiments. If the clustering of data are quite distinct (the class distance is the crucial standard of classification), then the k must be small. The key idea is we prefer the least k in the case that d % is bigger the threshold value.

The k-NN method gathers the nearest k neighbors and let them vote—the class of most neighbors wins. Theoretically, the more neighbors we consider, the smaller error rate it takes place. The general case is a little more complex. But by imagination, it is true to be the more

k the lower upper bound asymptotic to PBayes(e) if N is fixed.

One can use such algorithm to classify and cross validate a given cohort of samples based on the genes presented by this invention in Table 1. Most preferably the classification shall be performed based on the expression levels of the genes presented in Table 1 but may also combined with clinicopathological data as fare a they are measured in a continues manner (e.g. immune histo chemistry data, scoring date such as TNM status or biochemical properties of such tumor tissue.

With k=3 and >100 iteration one can get classifications as depicted below for a cross-validation experiment with the two classes “metastasizing breast tumors” (sample group 2) or “non-metastasizing breast tumors” (sample group 3. Affinities ranging from −1 to 1 for a given class (see Table 4).

TABLE 4

| | | | | | PREDICTED | PRE-DICTED | |
|---|---|---|---|---|---|---|---|
| | | | | | SAMPLE | SAMPLE | |
| EXPERIMENT | AGENT | CYCLES | DOSAGE | METASTASIS | GROUP 2 | GROUP 3 | CLASSIFICATION |
| Sample 1 | CMF | 6 × 2 | 500/40/600 | pos | 1 | −1 | true |
| Sample 2 | CMF | 6 × 2 | 500/40/600 | pos | 1 | −1 | true |
| Sample 3 | CMF | 6 × 2 | 500/40/600 | pos | 1 | −1 | true |
| Sample 4 | CMF | 6 × 2 | 500/40/600 | pos | 1 | −1 | true |
| Sample 5 | CMF | 6 × 2 | 500/40/600 | pos | −1 | 1 | false |
| Sample 6 | CMF | 6 × 2 | 500/40/600 | pos | 1 | −1 | true |
| Sample 7 | CMF | 6 × 2 | 500/40/600 | pos | 1 | −1 | true |
| Sample 8 | CMF | 6 × 2 | 500/40/600 | pos | 0.3548 | −0.3548 | no classification |
| Sample 9 | CMF | 6 × 2 | 500/40/600 | pos | 1 | −1 | true |
| Sample 10 | CMF | 6 × 2 | 500/40/600 | pos | 1 | −1 | true |
| Sample 11 | CMF | 6 × 2 | 500/40/600 | pos | 1 | −1 | true |
| Sample 12 | CMF | 6 × 2 | 500/40/600 | pos | 1 | −1 | true |
| Sample 13 | CMF | 6 × 2 | 500/40/600 | neg | −1 | 1 | true |
| Sample 14 | CMF | 6 × 2 | 500/40/600 | neg | 1 | −1 | false |
| Sample 15 | CMF | 6 × 2 | 500/40/600 | neg | −1 | 1 | true |
| Sample 16 | CMF | 6 × 2 | 500/40/600 | neg | −1 | 1 | true |
| Sample 17 | CMF | 6 × 2 | 500/40/600 | neg | −0.1852 | 0.1852 | no classification |
| Sample 18 | CMF | 6 × 2 | 500/40/600 | neg | −1 | 1 | true |
| Sample 19 | CMF | 6 × 2 | 500/40/600 | neg | −1 | 1 | true |
| Sample 20 | CMF | 6 × 2 | 500/40/600 | neg | 0.8333 | −0.8333 | no classification |
| Sample 21 | CMF | 6 × 2 | 500/40/600 | neg | −1 | 1 | true |
| Sample 22 | CMF | 6 × 2 | 500/40/600 | neg | −1 | 1 | true |
| Sample 23 | CMF | 6 × 2 | 500/40/600 | neg | −1 | 1 | true |
| Sample 24 | CMF | 6 × 2 | 500/40/600 | neg | −1 | 1 | true |
| Sample 25 | CMF | 6 × 2 | 500/40/600 | neg | −1 | 1 | true |
| Sample 26 | CMF | 6 × 2 | 500/40/600 | neg | −1 | 1 | true |
| Sample 27 | CMF | 6 × 2 | 500/40/600 | neg | 1 | −1 | false |
| Sample 28 | CMF | 6 × 2 | 500/40/600 | neg | −1 | 1 | true |
| Sample 29 | CMF | 6 × 2 | 500/40/600 | neg | −0.7647 | 0.7647 | no classification |
| Sample 30 | CMF | 6 × 2 | 500/40/600 | neg | −1 | 1 | true |
| Sample 31 | CMF | 6 × 2 | 500/40/600 | neg | −1 | 1 | true |
| Sample 32 | CMF | 6 × 2 | 500/40/600 | neg | −1 | 1 | true |
| Sample 33 | CMF | 6 × 2 | 500/40/600 | neg | −1 | 1 | true |
| Sample 34 | CMF | 6 × 2 | 500/40/600 | neg | −1 | 1 | true |
| Sample 35 | CMF | 6 × 2 | 500/40/600 | neg | −1 | 1 | true |
| Sample 36 | CMF | 6 × 2 | 500/40/600 | neg | −1 | 1 | true |

The misclassification of some samples or not classifiable samples may be due to low tumor amount in specimen.

The process of model generation and crossvalidation of predictive gene sets may follow the path outlined in FIG. 2, wherein a given cohort of samples is subdivided into two sets a so called training and a test set. Based on such training set genes can be picked and a preliminary model can be evaluated, further such model can be validated with the sample taken from the test set cohort. These two independent classifications of samples will lead to a final model (e.g. KNN algorithm and matrix) which can be further applied to new independent tumor samples.

### EXAMPLE 5

In order to get the most accurate prediction for response to chemotherapy based on the expression levels of genes listed in Table 1. One can implement a step wise classification model (e.g. decision tree) identifying first those individuals (tumor tissues) with the highest affinity (e.g. by k-NN classification) to the class of non metastasizing tumors (good prognosis group; sample group 3). If an so far unclassified tumor sample did not belong to this class on may perform a second classification step for this sample using the expression levels of the genes from Table 1 and some of the established clinicopahtological parameters such as hormone receptor status, age, TNM classification and risk criteria as established at the St. Gallen consensus conference or the NHI consensus conferences. Nevertheless a classification by the genes listed in Table 1 is sufficient to identify all patients at high risk for recurrence and/or distant metastasis.

### REFERENCES

### Patents Cited

U.S. Pat. No. 4,683,202
U.S. Pat. No. 5,593,839
U.S. Pat. No. 5,578,832
U.S. Pat. No. 5,556,752
U.S. Pat. No. 5,631,734
U.S. Pat. No. 5,599,695
U.S. Pat. No. 4,683,195
U.S. Pat. No. 6,203,987
WO 97/29212
WO 97/27317
WO 95/22058
WO 97/02357
WO 94/13804
WO 97/14028
EP 0 785 280
EP 0 799 897
EP 0 728 520
EP 0 721 016

### Other References Cited

(1) Publications cited: WHO. International Classification of Diseases, 10^{th} edition (ICD-10). WHO
(2) Sabin, L. H., Wittekind, C. (eds): TNM Classification of Malignant Tumors. Wiley, New York, 1997
(3) Sorlie et al., Proc Natl Acad Sci USA. 2001 Sep. 11; 98(19):10869-74 (3);
(4) van 't Veer et al., Nature. 2002 Jan. 31; 415(6871):530-6. (4).
(5) Perez, E. A.: Current Management of Metastatic Breast Cancer. Semin. Oncol., 1999; 26 (Suppl. 12): 1-10
(6) Sambrook et al., MOLECULAR CLONING: A LABORATORY MANUAL, 2d ed., 1989
(7) Ausubel et al., CURRENT PROTOCOLS IN MOLECULAR BIOLOGY, John Wiley & Sons, New York, N.Y., 1989.
(8) Tedder, T. F. et al., Proc. Natl. Acad. Sci. U.S.A. 85:208-212, 1988
(9) Hedrick, S. M. et al., Nature 308:149-153, 1984
(10) Bonner et al., J. Mol. Biol. 81, 123 1973
(11) Bolton and McCarthy, Proc. Natl. Acad. Sci. U.S.A. 48, 1390 1962
(12) Hampton et al., SEROLOGICAL METHODS: A LABORATORY MANUAL, APS Press, St. Paul, Minn., 1990
(13) Kohler et al., Nature 256, 495-497, 1985
(14) Takeda et al., Nature 314, 452-454, 1985
(15) Burton, Proc. Natl. Acad. Sci. 88, 11120-11123, 1991
(16) Thirion et al., Eur. J. Cancer Prev. 5, 507-11, 1996
(17) Coloma & Morrison, Nat. Biotechnol. 15, 159-63, 1997
(18) Mallender & Voss, J. Biol. Chem. Xno9, 199-206, 1994
(19) Verhaar et al., Int. J. Cancer 61, 497-501, 1995
(20) Orlandi et al., Proc. Natl. Acad. Sci. 86, 3833-3837, 1989
(21) Faneyte et al., Br J Cancer, 88:406-412, 2003.
(22) Perou et al., Nature, 406:747-752. 2000.
(23) Sorlie et al., Proc Natl Acad Sci USA, 100:8418-8423,
(24) Pusztai et al., Clin Cancer Res., 9:2406-2415, 2003.
(25) Ahr et al., J. Pathol., 195:312-320, 2001.
(26) Martin et al., Cancer Res., 60:2232-2238, 2000.
(27) van de Rijn et al., Am J. Pathol., 161:1991-1996, 2002.
(28) Huang et al., Lancet, 361:1590-1596, 2003.
(29) West et al., Proc Natl Acad Sci USA, 98:11462-11467, 2001
(30) van de Vijver et al., N Engl J. Med. 347:1999-2009, 2002.
(31) Sotiriou et al., Breast Cancer Res., 4:R3, Epub 2002 Mar. 20.
(32) Chang et al., Lancet, 362:362-369, 2003.
(33) Korn et al., Br J Cancer, 86:1093-1096, 2002.

1. Method for predicting therapeutic success of a given mode of treatment in a subject having breast cancer, comprising
(i) determining the pattern of expression levels of at least 6, 8, 10, 15, 20, 30, or 84 marker genes, comprised in the group of marker genes listed in Table 1,
(ii) comparing the pattern of expression levels determined in (i) with one or several reference pattern(s) of expression levels,
(iii) predicting therapeutic success for said given mode of treatment in said subject from the outcome of the comparison in step (ii).

2. Method of claim 1, wherein said given mode of treatment
(i) acts on cell proliferation, and/or
(ii) acts on cell survival, and/or
(iii) acts on cell motility; and/or
(iv) comprises administration of a chemotherapeutic agent.

3. Method of claim 1 or 2, wherein said given mode of treatment is CMF (cyclophosphamide, methotrexate, fluorouracil) chemotherapy.

4. Method of any of claims 1 to 3, wherein a predictive algorithm is used.

5. Method of treatment of a neoplastic disease in a subject, comprising
(i) predicting therapeutic success for a given mode of treatment in a subject having breast cancer by the method of any of claims 1 to 4,
(ii) treating said neoplastic disease in said patient by said mode of treatment, if said mode of treatment is predicted to be successful.

6. Method of selecting a therapy modality for a subject afflicted with a neoplastic disease, comprising
(i) obtaining a biological sample from said subject,
(ii) predicting from said sample, by the method of any of claims 1 to 4, therapeutic success in a subject having breast cancer for a plurality of individual modes of treatment,
(iii) selecting a mode of treatment which is predicted to be successful in step (ii).

7. Method of any of claims 1 to 6, wherein the expression level is determined
(i) with a hybridization based method, or
(ii) with a hybridization based method utilizing arrayed probes, or
(iii) with a hybridization based method utilizing individually labeled probes, or
(iv) by real time real time PCR, or
(v) by assessing the expression of polypeptides, proteins or derivatives thereof, or
(vi) by assessing the amount of polypeptides, proteins or derivatives thereof.

8. A kit comprising at least 6, 8, 10, 15, 20, 30, or 84 primer pairs and probes suitable for marker genes comprised in the group of marker genes listed in Table 1.

9. A kit comprising at least 6, 8, 10, 15, 20, 30, or 84 individually labeled probes, each having a sequence complementary to any of sequences listed in Table 1.

10. A kit comprising at least 6, 8, 10, 15, 20, 30, or 84 arrayed probes, each having a sequence complementary to any of the sequences listed in Table 1.
